(19) United States
(12) Patent Application Publication
(10) Pub. No.: US 2025/0277794 A1
(43) Pub. Date: Sep. 4, 2025

(54) SQSTM1 AND ITS USE IN CANCER THERAPY

(71) Applicants: CENTRE NATIONAL DE LA RECHERCHE SCIENTIFIQUE, Paris (FR); Institut National de la Santé Et de la Recherche Médicale, Paris cedex 13 (FR); UNIVERSITÉ CÔTE D'AZUR, Nice (FR)

(72) Inventors: Baharia MOGRABI, Nice (FR); Nathalie YAZBECK, Vigo Pontevedra (ES); Amine BELAID, Senlis (FR); Grégoire D'ANDREA, Nice (FR); Iris GROSJEAN, Parempuyre (FR); Paul HOFMAN, Falicon (FR)

(21) Appl. No.: 18/266,713

(22) PCT Filed: Dec. 15, 2021
(86) PCT No.: PCT/EP2021/085911

§ 371 Date: Jun. 12, 2023

(30) Foreign Application Priority Data
Dec. 15, 2020 (EP) 20306577.6

(51) Int. Cl. G01N 33/574; A61K 38/17; A61K 45/06

(52) U.S. Cl. CPC G01N 33/57484; A61K 38/1709; A61K 45/06

(57) ABSTRACT

The invention relates to the use of a SQSTM1/p62 protein for modulating and predicting the response to: —an immunotherapy against immune checkpoint inhibitors: or—a combination of an immunotherapy, preferably an ICI, and a chemotherapy

### CROSS-REFERENCE TO RELATED PATENT APPLICATIONS

This application is a U.S. National Stage Application under 35 U.S.C. § 371 of International Patent Application No. PCT/EP2021/085911 filed Dec. 15, 2021, which claims the benefit of priority of European Patent Application No. 20306577.6 filed Dec. 15, 2020, both of which are incorporated by reference in their entireties. The International Application was published on Jun. 23, 2022, as International Publication No. WO/2022/129180 A1.

### SEQUENCE LISTING

The instant application contains a Sequence Listing which has been submitted electronically in ASCII format and is hereby incorporated by reference in its entirety. Said ASCII copy, created on Jun. 12, 2023, is named AS FILED—BR 147115—Sequence Listing—12286/011952-US0 and is 16,384 bytes in size.

The invention relates to the protein SQSTM1 and its use in therapy.

The immune system in higher eukaryotes plays a central role against invasion by foreign pathogens and infected cells. In the absence of external insults, the immune system also carefully surveys and efficiently recognizes nascent transformed cells by detecting unusual antigens or aberrantly overexpressed proteins at the cell surface.

Classically, innate immune cells-natural killer cells (NKs), dendritic cells (DCs), polymorphonuclear leukocytes, and macrophages—are recruited as the first line of defense to attack the transformed cells by cytolytic killing. Then, antigen-presenting cells (APCs), such as DCs, process the tumor antigens and migrate to the lymphatic nodes to prime a more focused adaptive immune response mediated by B and T lymphocytes. Upon antigen presentation through MHC-II and MHC-I receptors to naïve CD4+ and CD8+ T cells, respectively, these cells undergo clonal expansion and differentiation to exert effector or memory functions. T cell functions are dictated at the molecular level by the TCR-dependent and cytokine-dependent signaling cascades culminating in the nucleus and activating lineage-specific transcription factors. This results in the development of a broad panel of T lymphocytes exerting diverse functions and characterized by specific surface and nuclear markers, as well as secreted effector molecules.

Of the T cell subsets, the “serial killers” are clearly the CD8+ cytolytic T lymphocytes (CTLs) that migrate to the tumor site and cooperate with Th1 and Th2 CD4+ helpers to attack and kill the transformed cells. While the CD4+ cells “help” by creating an immuno-stimulatory environment, the CTLs induce apoptotic death in target cells by secreting Fas ligand and proinflammatory tumor-necrosis factor (TNF), as well as cytotoxic granules containing Perforin (a pore-forming protein) and Granzymes (serine proteases). Of chemokines, numerous studies emphasize the importance of IFN-γ in tumor eradication. This cytokine is released by activated CD4 and CD8 T cells and controls APC's development and functions. The apoptotic tumor cells are then rapidly detected and engulfed by professional phagocytes, such as macrophages and the dendritic cells, to present the tumor antigens to CTL and avoid excessive inflammation. In addition, CD4+ regulatory T cells (Treg) secrete transforming growth factor β (TGF-β), IL-10, and IL-35 that suppress the pro-inflammatory response, thereby limiting tissue damage. Ideally, a tuned balance between cytotoxic and regulatory activities allows tumor cell removal and preserves the integrity of the surrounding healthy tissue.

In theory, an effective immune surveillance would yield a successful T cell priming and tumor eradication.

However, clinically diagnosed tumors attest to the ability of neoplastic cells to escape immune surveillance. Of immunosuppressive strategies, the T cell coinhibitory pathways are hijacked by tumor cells to circumvent the anti-tumor responses. During inflammation, these co-inhibitory pathways (also termed “immune checkpoints”) are expressed on immune and epithelial cells to balance the co-stimulatory signals, inhibit T cell functions, and avoid excessive cytotoxicity.

Programmed death-1 (PD-1) and its ligands PD-L1 and PD-L2 form by far the most famous immune inhibitory couples. PD-L1 is overexpressed at the cell surface of various cancers: melanoma, glioma, lung, colon, pancreas, breast, gut, kidney, bladder, and ovary cancers, and is associated with poor overall survival. When bound to its ligand PD-L1, PD-1 signaling reduces T cell activation (IFN-γ production), glycolysis, and cell cycle progression. Although the signaling events underlying this reprogramming remain to be explored, it is now clear that the resulting dysfunctional T cells can be reinvigorated by blocking the PD-1/PD-L1 interaction.

Knowledge of these checkpoints has led scientists to use them to develop new therapy against tumors: the checkpoint inhibitors immunotherapies.

Immune checkpoint inhibitors (ICI) have proven effective in treating several advanced cancers and prolonging the overall survival, particularly the PD-1/PD-L1 blocking antibodies that reinvigorate tumor-infiltrating lymphocytes (TILs) and constitute a valuable weapon against tumor development.

Unfortunately, despite the highest clinical benefit observed in cancer patients, with nearly 87% objective response in Hodgkin's disease and 70% in desmoplastic melanoma, the vast majority of the other cancers such as lung cancers and melanoma experienced at best 25-30% efficacy to checkpoint inhibitors immunotherapies. The resulting 80% of cancer patients suffer from innate or acquired resistance to ICI.

So there is a need to provide a biomarker that could help the pathologist determine if a checkpoint inhibitor immunotherapy will be efficient or not on a determined tumor.

The purpose of the invention is, therefore, to obviate this lack of the prior art.

One aim of the invention is to provide the use of a known protein as a predictive marker of the efficacy of a checkpoint inhibitor immunotherapy.

One other aim of the invention is to provide a method for predicting or efficiently treating tumors that could be resistant to this kind of immunotherapy.

Another aim of the invention is to provide a simple and ready-to-use kit to determine if a tumor will or will not respond to immunotherapy alone or in combination. Thus, the invention relates to the use of a SQSTM1, also called p62 or called SQSTM1/p62 protein for modulating, preferably in vitro, the response to:
an immunotherapy preferably an immunotherapy against immune checkpoint inhibitors, also called ICI;
a chemotherapy using an agent which is not an agent interfering with DNA repair; or
a combination of an immunotherapy, preferably an ICI, and a chemotherapy of a cell of a tumor.

The invention is based on the unexpected observation made by the inventors that SQSTM1/p62 is a central marker of the response to anti-cancer therapy. Thus by modulating the expression of SQSTM1/p62 protein, it is possible to modify or modulate the response of a cell of a tumor to an immunotherapy, a chemotherapy using an agent which is not an agent interfering with DNA repair, or a combination of an immunotherapy and a chemotherapy.

This means advantageously that in vitro a cell of tumor could respond differently to a treatment with the above therapy when SQSTM1 is present of absent or at a different level.

The invention relates to a composition comprising a SQSTM1/p62 protein for modulating, preferably in vitro,
an immunotherapy, preferably an immunotherapy against immune checkpoint inhibitors, also called ICI;
a chemotherapy using an agent which is not an agent interfering with DNA repair; or
a combination of an immunotherapy, preferably an ICI, and a chemotherapy of a cell of tumor.

Famous for being the first autophagy adaptor protein, Sequestosome 1 protein or SQSTM1/p62, is most importantly a signaling hub controlling a myriad of cellular functions, including cell growth, cell migration, and cell survival. SQSTM1/p62 does not have an intrinsic signaling function but interacts with kinases, ubiquitin ligases, and other proteins to drive signaling pathways.

SQSTM1/p62 protein was reported to be deregulated in cancers, but it was never taught, nor suggested that SQSTM1/p62 protein could be a central marker for evaluating the resistance or the sensibility to anticancer therapies.

In the invention, it is meant by “chemotherapy,” either a treatment with chemotherapeutic compounds or a treatment by using radiotherapy.

A chemotherapeutic compound corresponds, according to the invention, to a compound affecting DNA damage, DNA repair, DNA replication, DNA methylation, epigenetic modifications, innate defense, IFN response or cell division in a cancer cell, but also in normal cells.

In the invention, an immunotherapy or cancer immunotherapy encompasses various forms, including targeted antibodies, cancer vaccines, adoptive cell transfer, tumor-infecting viruses, synthetic viruses, synthetic RNA/DNA, and immune checkpoint inhibitors, cytokines, and adjuvants. The objective of immunotherapy is to boost the body's natural defenses to fight cancer cells.

One of the specific immunotherapies is the use of inhibitors of immune checkpoints.

Immune checkpoints are a normal part of the immune system and prevent an immune response from being so strong that it destroys healthy cells in the body. Immune checkpoints engage when proteins on the T cell surface recognize and bind to partner proteins on other cells, such as some tumor cells. When the checkpoint and partner proteins bind together, they send an “off” signal to the T cells. This can prevent the immune system from destroying the cancer. Immunotherapy drugs, called immune checkpoint inhibitors, work by blocking checkpoint proteins from binding with their partner proteins. This prevents the “off” signal from being sent, allowing the T cells to kill cancer cells. One such drug acts against a checkpoint protein called CTLA-4 protein, PD-1 protein, or its partner protein PD-L1.

Advantageously, the invention relates to the use of a SQSTM1/p62 protein for modulating, preferably in vitro, the response to an immunotherapy against immune checkpoint inhibitors.

Advantageously, the invention relates to the use of a SQSTM1/p62 protein for modulating, preferably in vitro, the response to a chemotherapy using an agent that is not an agent interfering with DNA repair.

Advantageously, the invention relates to the use of a SQSTM1/p62 protein for modulating, preferably in vitro, the response to a combination of an ICI and a chemotherapy.

Advantageously, the invention relates to the use defined above, wherein said SQSTM1/p62 protein comprises or consists essentially or consists of the amino acid sequence as set forth in SEQ ID NO: 1.

Advantagesouly, the invention related to the above composition as defined above, for its use as defined above, wherein said SQSTM1/p62 protein comprises or consists essentially or consists of the amino acid sequence as set forth in SEQ ID NO: 1.

The human SQSTM1/p62 protein comprises or consists of the amino acid sequence as set forth in SEQ ID NO: 1, represented hereafter:

MASLIVKAYL LGKEDAAREI RRFSFCCSPE PEAEAEAAAG
PGPCERLLSR VAALFPALRP GGFQAHYRDE DGDLVAFSSD
EELTMAMSYV KDDIFRIYIK EKKECRRDHR PPCAQEAPRN
MVHPNVICDG CNGPVVGTRY KCSVCPDYDL CSVCEGKGLH
RGHTKLAFPS PFGHLSEGES HSRWLRKVKH GHFGWPGWEM
GPPGNWSPRP PRAGEARPGP TAESASGPSE DPSVNFLKNV
GESVAAALSP LGIEVDIDVE HGGKRSRLTP VSPESSSTEE
KSSSQPSSCC SDPSKPGGNV EGATQSLAEQ MRKIALESEG
RPEEQMESDN CSGGDDDWTH LSSKEVDPST GELQSLQMPE
SEGPSSLDPS QEGPTGLKEA ALYPHLPPEA DPRLIESLSQ
MLSMGFSDEG GWLTRLLQTK NYDIGAALDT IQYSKHPPPL.

This protein is coded by the nucleic acid molecule as referenced in the NCBI database NM_003900.5, and listed as SEQ ID NO: 2.

Therefore, in an advantageous embodiment, the invention relates to the use of a SQSTM1/p62 protein as set for the in SEQ ID NO: 1 for modulating, preferably in vitro, the response to an immunotherapy against immune checkpoint inhibitors.

Advantageously, the invention relates to the use of a SQSTM1/p62 protein as set for the in SEQ ID NO: 1 for modulating, preferably in vitro, the response to a chemotherapy using an agent, which is not an agent interfering with DNA repair

Advantageously, the invention relates to the use of a SQSTM1/p62 protein as set for the in SEQ ID NO: 1 for modulating, preferably in vitro, the response to a combination of an ICI, and a chemotherapy.

In one other aspect, the invention also relates to a method for predicting, preferably in vitro or ex vivo, the resistance to a therapy of a tumor, said therapy being a chemotherapy and/or an immunotherapy, said method comprising:
evaluating the presence or the absence or the amount of an SQSTM1/p62protein in a biological sample originating from the tumor,
comparing the presence, absence, or the amount of the SQSTM1/p62protein with the amount of the SQSTM1/p62protein in a control sample,
concluding that
the tumor will be likely to be resistant to the therapy when SQSTM1/p62 protein is absent or lower than or equal to the amount obtained in the control sample in the said biological sample, and
the tumor will likely be sensitive to the therapy when SQSTM1/p62 protein is present or higher than a control level in the said biological sample.

In the invention, the inventors have shown that the expression of, or the deregulation of the expression of the SQSTM1/p62 protein is a valuable marker of the resistance, or sensibility, to a therapy of a tumor.

Indeed, the inventors have shown that the presence, or an increase in expression of SQSTM1/p62 protein in a tumor or a tumoral sample is a marker of the sensibility of said tumor or said tumor sample to a chemotherapy, an immunotherapy or both therapies. In the same manner, when SQSTM1/p62 protein is not expressed, or unexpressed in a tumor or a tumoral, the tumor or the tumor sample will be resistant to a chemotherapy, an immunotherapy or both therapies.

Presence or absence, or variation of the amount of the protein, i.e., of SQSTM1/p62 protein, is determinant. Indeed, the biological effect of the presence of SQSTM1/p62 protein is the key point of the sensibility/resistance of a tumor. Therefore, it is not sufficient to evaluate the amount of nucleic acid molecules (i.e., RNA) coding for SQSTM1/p62, especially if there is no correlation between the transcription level and the translation level.

Detection of the presence or the amount of SQSTM1/p62 protein can be carried out by any technic known in the art to specifically detect protein, in particular by using immunological means such as antibodies or their derivates. This detection can be carried out by immunohistochemistry, immunoblotting, immunofluorescence in situ, or by using a flow cytometer.

The presence, absence, or variation of the amount of the SQSTM1/p62 protein is evaluated in a sample originating from a tumor, i.e., from a biopsy or a liquid biopsy, or from a blood sample in which are present circulating cells originating from a said tumor. In the case of a hematological tumor, such as leukemia or lymphomas, the biological sample is either a blood sample, or a biopsy obtained from bone marrow, or from an organ where malignant cells were engrafted.

The presence, absence or amount of SQSTM1/p62 is determined compared to the presence, absence, or amount of the protein in a reference sample. Advantageously, the reference sample is of the same nature or origin as the tumor. This means that if the tumor is a lung tumor, the reference sample will be obtained from the lung of an individual who is not affected by a lung cancer or the adjacent control tissues from the same patient.

The reference sample can be either a negative reference sample, i.e., a sample known to not correspond to a tumor, or a positive reference sample for which the amount or absence of the SQSTM1/p62 protein is known. The reference sample in the invention can be obtained, for instance, from adjacent healthy tissues.

In order to compare the presence, absence, or amount between both the sample to be studied and the reference sample, the detection of the SQSTM1/p62 protein can be quantified by means known in the art, such as quantification of luminescence or fluorescence, brown colorimetric signal. Detection of a punctate SQSTM1/p62-staining pattern in overexpressing cancers versus barely uniform staining in healthy tissue (see Example below).

Advantageously, the SQSTM1/p62 protein comprises or consists of the amino acid sequence as set forth in SEQ ID NO: 1.

So, advantageously, the invention also relates to a method for predicting the resistance to a therapy of a tumor, said therapy being a chemotherapy and/or an immunotherapy, said method comprising:
evaluating the presence or the absence or the amount of an SQSTM1/p62 protein as set forth in SEQ ID NO: 1 in a biological sample originating from the tumor,
comparing the presence, absence, or the amount of the SQSTM1/p62 protein with the amount of the SQSTM1/p62 protein as set forth in SEQ ID NO: 1 in a control sample,
concluding that
the tumor will be likely to be resistant to the therapy when SQSTM1/p62 protein as set forth in SEQ ID NO: 1 is absent or lower than or equal to the amount obtained in the control sample in the said biological sample, and
the tumor will be likely to be sensitive to the therapy when SQSTM1/p62 protein as set forth in SEQ ID NO: 1 is present or higher than a control level in the said biological sample.

Advantageously, the invention relates to the method as defined above, wherein the presence or absence of the SQSTM1/p62 protein is evaluated in situ in the biological sample, preferably in a tissue biopsy or a liquid biopsy.

As known in the art, a tissue biopsy involves the extraction of sample cells or tissues for examination to determine the presence or extent of a disease. The tissue is generally examined under a microscope by a pathologist; it may also be analyzed chemically or by using proteomic analysis.

A liquid biopsy corresponds to the analysis of tumors using biomarkers circulating in fluids such as the blood. There are several types of liquid biopsy methods; method selection depends on the condition that is being studied. Liquid biopsy is based on the detection of cancer cells, but also proteins and circulating tumor nucleic acids (DNA or RNA-ctDNA).

A wide variety of biomarkers may be studied to detect or monitor diseases.

Advantageously, the invention relates to the method as defined above, wherein the in situ evaluation is carried out by immunologic means.

As mentioned above, the presence, absence, or amount of SQSTM1/p62 protein is evaluated in situ in the biological sample, which means that the presence, absence, or amount of SQSTM1/p62 protein is evaluated directly on the cells originating from the tumor.

When a tissue biopsy is used, it is advantageous to carry out immunohistochemistry techniques to determine the presence or the absence or the amount of the protein. A specific anti-SQSTM1/p62 antibody is therefore used, commonly coupled with a secondary antibody coupled with a labeling chromogen such as peroxidase or alkaline phosphatase. Immunocytochemistry techniques can also be carried out.

When a liquid biopsy is used, it is advantageous to use a specific anti-SQSTM1/p62 antibody coupled to a fluorophore in order to detect either in the sample, or by using a flow cytometer the cells expressing the SQSTM1/p62 proteins. Secreted SQSTM1/p62 can also be detected by well-known technics, such as ELISA using anti SQSTM1/p62 antibody or Proteomic approach.

These techniques are well known in the art, and the skilled person, having knowledge in identifying proteins in cells, would use the most appropriate techniques depending upon the type of the sample to be studied.

In another aspect, the invention also relates to a method for predicting, preferably in vitro, the survival rate of a patient afflicted by a tumor, said method comprising:
Evaluating the presence or the absence or the amount of an SQSTM1/p62 protein in a biological sample originating from the tumor,
Comparing the presence, absence, or the amount of the SQSTM1/p62 protein with the amount of the SQSTM1/p62 protein evaluated in a control sample,
concluding that
when SQSTM1/p62 in the said biological sample is absent or lower than or equal to the amount obtained in the control sample, then the patient will have a survival rate higher than 80% after five years, and
when SQSTM1/p62 in the said biological sample is present or higher than the amount obtained in the control sample, then the patient will have a survival rate lower than 70% after five years.

In another aspect of the invention, the inventors have identified that it is possible to predict the survival rate of a patient afflicted by a tumor, by merely measuring the expression level of the protein SQSTM1/p62 as disclosed above.

The inventors noted that when the expression level of the protein SQSTM1/p62 is lower than or equal to the relative expression level observed in a non-tumoral, and more generally non-healthy biological sample, then the individual from which the sample is obtained will have a likelihood of survival over five years which is higher than 80%. On the contrary, when the expression level, i.e., the amount, of the protein is higher than the level in a reference sample, then the individual from which the sample is obtained will have a likelihood of survival over five years, which is lower than 80%.

Therefore, the inventors have identified that an increased expression of SQSTM1/p62 protein in a tumor is a hallmark of the severity of a tumor and its potential aggressivity, for instance, due to tumor immune evasion.

Therefore, assessing the amount of the variation of the amount of SQSTM1/p62 protein in a biological tumor sample can be useful for determining the outcome of the patient, and to provide him/her an appropriate therapy.

Advantageously, the invention relates to the method as defined above, wherein the presence, absence, or amount of
a PD-L1 protein, and
T CD8 lymphocytes
are evaluated concomitantly to the presence or the absence or the amount of an SQSTM1/p62protein, and compared with the presence, absence, or the amount of the respective PD-L1 protein and T CD8 lymphocytes evaluated in a control sample, and wherein
when SQSTM1/p62 protein, PD-L1 protein, and T CD8 lymphocytes in the said biological sample are present or higher than the amount obtained in the control sample, the patient will have a survival rate lower than 50% after five years.

The inventors have also identified advantageously that assessing further the amount of PD-L1 protein and T CD8 lymphocyte can be useful in order to refine the prognosis of survival over five years. Indeed, the increase in SQSTM1/p62 and PD-L1 proteins, along with the increase in T CD8 lymphocytes, allows the partitioner to forecast a bad outcome of the tumor over five years. Therefore, he could propose an appropriate therapy, such as immunotherapy using anti-PD-L1 antibodies, associated possibly with other compounds.

In the invention, the above mentioned patients can be further treated with DNA damage agents such as cisplatin, docetaxel, oxaliplatin, DNA/epigenetic drugs (notably DNA methylase inhibitors, 5-azacytidine, decitabine, HDAC inhibitors, histone methylase inhibitors), cell cycle inhibitors (CDK4/6 inhibitors such as palbociclib/PD-0332991, Abemaciclib, and ribociclib/LEE011), innate defense, IFN response.

The invention also relates to a method for predicting, preferably in vitro, the survival rate of a patient afflicted by a tumor and treated with a chemotherapy and/or an immunotherapy, said method comprising:
evaluating the presence or the absence or the amount of an SQSTM1/p62 protein in a biological sample originating from the tumor,
comparing the presence, absence, or the amount of the SQSTM1/p62 protein with the amount of the SQSTM1/p62 protein evaluated in a control sample,
concluding that
when SQSTM1/p62 in the said biological sample is absent or lower than or equal to the amount obtained in the control sample, then the patient will have a survival rate lower than or equal to 10% after 20 months of treatment, and
when SQSTM1/p62 in the said biological sample is present or higher than the amount obtained in the control sample, then the patient will have a survival rate equal to or higher than 50% after 20 months of treatment.

The invention also relates to a method for predicting, preferably in vitro, the survival rate of a patient afflicted by a tumor and treated with a chemotherapy or an immunotherapy, or both, said method comprising:
evaluating the presence or the absence or the amount of an SQSTM1/p62 protein in a biological sample originating from the tumor,
comparing the presence, absence, or the amount of the SQSTM1/p62 protein with the amount of the SQSTM1/p62 protein evaluated in a control sample,
concluding that
when SQSTM1/p62 in the said biological sample is absent or lower than or equal to the amount obtained in the control sample, then the patient will have a survival rate lower than or equal to 10% after 20 months of treatment, and
when SQSTM1/p62 in the said biological sample is present or higher than the amount obtained in the control sample, then the patient will have a survival rate equal to or higher than 50% after 20 months of treatment.

In another aspect of the invention, the inventors have also identified that the prediction of the survival rate of a patient afflicted by a tumor and treated by using an immunotherapy or chemotherapy can be assessed by measuring the expression level of the SQSTM1/p62 protein.

The inventors have noted that when a patient with a tumor and treated with an immunotherapy or a chemotherapy, or both, have an amount of SQSTM1/p62 higher than a reference amount, then the patient with have a good outcome over 20 months. However, when the amount of SQSTM1/p62 is lower than a reference level, the patient will have a bad outcome over 20 months.

It is therefore possible to enforce the expression of SQSTM1/p62 in the tumor of said patients, in order to intend to increase the outcome. Vector therapies, or chemical therapies can be used, in order to increase the amount of SQSTM1/p62 protein. The skilled person could easily determine the best way to enforce such protein expression.

The invention also relates to a composition comprising:
an SQSTM1/p62 protein, or
a nucleic acid molecule coding for said SQSTM1/p62 protein;
along with an immunotherapeutic antibody directed against a checkpoint inhibitor or a combination or a combination of a chemotherapeutic agent and an immunotherapeutic antibody directed against a checkpoint inhibitor,
for its use for treating pathology involving inflammation.

In another aspect, the invention relates to a composition comprising an SQSTM1/p62 protein,
along with a chemotherapeutic agent or an immunotherapeutic antibody directed against a checkpoint inhibitor, or both,
for its use for treating pathology involving inflammation.

Moreover, the invention relates to a composition comprising a nucleic acid molecule coding for said SQSTM1/p62 protein;
along with a chemotherapeutic agent or an immunotherapeutic antibody directed against a checkpoint inhibitor, or both,
for its use for treating pathology involving inflammation.

In another aspect, the invention relates to a composition comprising one of the effectors of SQSTM1/p62;
along with a chemotherapeutic agent or an immunotherapeutic antibody directed against a checkpoint inhibitor, or both,
for its use for treating pathology involving inflammation.

In the invention, a pathology involving inflammation can be, for instance, a pathology involving cancers, and autoimmune diseases, as well as infections.

Moreover, the invention relates to a composition comprising a nucleic acid molecule coding for one of the effectors of SQSTM1/p62;
along with a chemotherapeutic agent or an immunotherapeutic antibody, or both,
for its use for treating pathology involving inflammation.

As mentioned above, the inventors have unexpectedly found that an increased expression of SQSTM1/p62 protein may significantly reduce the development or progression of inflammation and pathologies involving inflammation.

The above composition may contain either protein itself, in particular the protein consisting of the amino acid sequence as set forth in SEQ ID NO: 1, or a nucleic acid molecule coding for the said protein.

The above mentioned a nucleic acid molecule may be the molecule as set forth in SEQ ID NO: 2.

Advantageously, the SQSTM1/p62 protein or the nucleic acid molecule coding for said protein, and the chemotherapeutic agent and/or the immunotherapeutic antibody, can be used simultaneously, separately or sequentially, at a determined dosage determined by the skilled person. The separated or sequential use will depend upon the compatibility between the protein and the chemotherapeutic agent and/or the immunotherapeutic antibody.

Advantageously, the invention relates to the composition as defined above for its use as defined above, wherein said pathology involving inflammation are cancers, in particular primary tumors or metastatic tumors.

A primary tumor is a tumor growing at the anatomical site where tumor progression began and proceeded to yield a cancerous mass. Most cancers develop at their primary site.

A metastatic tumor is a tumor that has spread from the part of the body where it started (the primary site) to other parts of the body. When cancer cells break away from a tumor, they can travel to other parts of the body through the bloodstream or the lymph system.

Advantageously, the invention relates to the composition as defined above for its use as defined above, wherein said cancers are lung cancers, kidneys cancers, bladder cancers, head neck cancers, uterine cancer, melanoma, Hodgkin's lymphoma, Large B cell lymphoma, Merkel disease, hepatocellular carcinoma, and gastrointestinal cancers, preferably gastro-intestinal cancer with minisatellite instability.

Merkel cell carcinoma is a rare type of skin cancer that usually appears as a flesh-colored or bluish-red nodule, often on your face, head, or neck. Merkel cell carcinoma is also called neuroendocrine carcinoma of the skin

The invention also relates to a kit comprising:
an SQSTM1 protein, or a nucleic acid molecule coding for said SQSTM1 protein or said fragment thereof,
an antibody useful for inducing immunotherapy directed against a check-point inhibitor, and
a chemotherapeutic agent.

A kit according to the invention advantageously contains SQSTM1 protein as set forth in SEQ ID NO: 1, or a nucleic acid molecule coding for the SQSTM1 protein, the nucleic acid molecule being advantageously the molecule as set forth in SEQ ID NO: 2.

The kit according to the above definition, wherein said antibody is an anti-PD-L1 antibody, an anti-PD-1 antibody, or an anti-CTLA-4 antibody.

Advantageously, the invention also relates to a kit comprising:
an SQSTM1 protein, or a nucleic acid molecule coding for said SQSTM1 protein or said fragment thereof,
anti-PD-L1 antibody, an anti-PD-1 antibody, or an anti-CTLA-4 antibody useful for inducing immunotherapy, and
a chemotherapeutic agent.

Advantageously, the invention relates to the kit as defined above, wherein said chemotherapeutic is either a chemotherapeutic containing platin compounds, or a Paclitaxel or Docetaxel compound, or a radiotherapy.

Examples of compounds containing platin are, for instance, cisplatin, carboplatin, oxaliplatin, nedaplatin, triplatin tetranitrate, phenanthriplatin, satraplatin, or picoplatin. Other compounds such as DNA methylase inhibitors, 5-azacytidine, decitabine HDAC inhibitors, histone methylase inhibitors), cell cycle inhibitors (CDK4/6 inhibitors such as palbociclib/PD-0332991, Abemaciclib, and ribociclib/LEE011), innate defense, IFN response can also be used.

Radiotherapy may be used in the early stages of cancer or after it has started to spread. The most common types are:
external radiotherapy, where a machine is used to aim beams of radiation at the tumor carefully;
radiotherapy implants (brachytherapy), where small pieces of radioactive metal are (usually temporarily) placed inside your body near the tumor;
radiotherapy injections, capsules, or drinks (radioisotope therapy), where you swallow a radioactive liquid, or have it injected into your blood; and
intrabeam radiotherapy, where radiation is delivered directly at the tumor during breast cancer surgery.

The amount of radiation used in radiotherapy is measured in grays (Gy) and varies depending on the type and stage of the cancer being treated. The typical dose for a solid epithelial tumor ranges from 60 to 80 Gy for curative cases, while lymphomas are treated with 20 to 40 Gy.

Preventive doses are typically around 45-60 Gy in 1.8-2 Gy fractions (for breast, head, and neck cancers). Many other factors are considered by radiation oncologists when selecting a dose, including whether the patient is receiving chemotherapy, patient comorbidities, whether radiation therapy is being administered before or after surgery, and the degree of surgery success.

The invention will be better understood in view of the following drawings and the example below.

The invention also relates to a method for treating an individual afflicted by a tumor, said method comprising the following steps
Evaluating the presence or the absence or the amount of an SQSTM1/p62 protein in a biological sample originating from the tumor,
Comparing the presence, absence, or the amount of the SQSTM1/p62 protein with the amount of the SQSTM1/p62 protein evaluated in a control sample,
concluding that
when SQSTM1/p62 protein in the said biological sample is present or higher than or equal to the amount obtained in the control sample, then an immunotherapy, preferably an immunotherapy against immune checkpoint inhibitors, is administered to the patient, in association or not with a chemotherapy, in particular DNA damage inducing agent; and
when SQSTM1/p62 in a said biological sample is absent or lower than the amount obtained in the control sample, then an immunotherapy, preferably an immunotherapy against immune checkpoint inhibitors, is administered to the patient, in association with a member of the taxane-based chemotherapies.

The invention also relates to a composition comprising at least an immunotherapeutic compound, possibly in association with a chemotherapeutic agent, for its use for treating patients afflicted by a tumor the cells of which having an expression of SQSTM1/p62 protein, or having an amount of SQSTM1/p62 protein higher than a control sample.

The invention also relates to a composition comprising at least an immunotherapeutic compound, association with taxanes, for its use for treating patients afflicted by a tumor the cells of which having no expression of SQSTM1/p62 protein, or having an amount of SQSTM1/p62 protein lower than a control sample.

In other words, the invention relates to a composition comprising an immunotherapeutic compound in association with taxanes, for its use for treating tumors that do not express SQSTM1/p62 protein, or tumors that express SQSTM1/p62 protein at a level lower than than the level of SQSTM1/p62 protein in a control tissue.

The compositions mentioned above that are used in order to treat specific cancers preferably contains an anti-PD-L1 antibody, an anti-PD-1 antibody, or an Anti-CTLA-4 antibody. This also apply to the above-described method of treatment.

In other words, the invention relates to a composition comprising an immunotherapeutic compound in association with DNA damage-inducing agent, for its use for treating tumors that express SQSTM1/p62 protein, or tumors that express SQSTM1/p62 protein at a level higher than the level of SQSTM1/p62 protein in a control tissue.

### BRIEF DESCRIPTION OF THE FIGURES

FIGS. 1 to 3: Resistances to cancer therapies share a DNA damage repair signature and a “COLD” immunogenic profile

FIG. 1 represents a GSEA analysis showing that there is significant activation of the gene set of DNA repair in Cold human melanomas (SKCM) (negative for CD8A, and negative for HLA-B transcript levels) from The Cancer Genome Atlas database (TCGA, PanCancer Atlas, up) and corresponding Heatmap (bottom). Key color: The light grey and dark grey colors correspond respectively to low and high expressions

FIG. 2 represents GSEA plots showing a significant activation of the gene set of DNA repair and inhibition of allograft rejection, leukocyte mediated cytotoxicity genesets in Cisplatin resistant non-small cell lung cancer (NSCLC, Responders-R, Non-Responders-NR, GEO prospect study).

FIG. 3 represents GSEA and box-plots analyses showing significant enrichment of DNA repair and cell cycle checkpoint genesets in ICI resistant melanoma (anti-PD-1, NR, cBioportal, https://portals.broadinstitute.org/single-cell/study/melanoma-immunotherapy-resistance #study-visualize, GSE115978).

FIGS. 4 to 9: SQSTM1/p62 is a powerful predictor of response to immunotherapies.

FIG. 4 represents that SQSTM1 is at the intersection of the Venn diagram of the differential expressed genes between “response to ICI,” “response to RT,” (RT: Radiotherapy) and “NF-kB signaling” signatures (GSEA, KEGG).

FIG. 5 represents the structure and interacting partners of p62/SQSTM1. SQSTM1 is composed of multiple domains required for its interaction with autophagic machinery and with signaling pathways involved in cell death, inflammation, DNA repair, and, ultimately cancer. PB1 Phox and Bem1; ZZ Zinc finger; RIR Raptor Interacting Region; TBS Traf6 Biding Site; LIR Lc3 Interacting Region; KIR Keap1 Interacting Region; UBA Ub-Associated; NLS Nuclear Localisation Signal; NES Nuclear Export Signal.

FIG. 6 represents a GSEA plot of antigenic presentation and DNA repair signatures positively and negatively correlated to SQSTM1 transcript levels in human melanomas (SKCM) and lung cancers (LUAD) from The Cancer Genome Atlas database (TCGA, PanCancer Atlas).

FIG. 7 represents lists of the most differentially expressed signaling scaffold proteins between ICI responders (R) versus non-responders (NR). Inset. Boxplot of SQSTM1 mRNA expression in ICI responders (R) versus non-responders (NR) (anti-PD-1, adjusted p-value, melanoma, GSE115978).

FIG. 8 represents Kaplan-Meier plots showing the disease-specific survival (DSS) curves in SQSTM1 high (H) vs. low (L) and in SQSTM1 high/PD-L1 high/CD8 high (HHH) vs. other groups of patients treated with immunotherapy.

FIG. 9 represents representative images of SQSTM1, PD-L1, and CD8 positive and negative IHC staining on LUAD tumor sections. Note that a single SQSTM1 assay could accurately discriminate between the “False negative” (high SQSTM1 expression) cases that respond to immunotherapy and the “False positive” cases (non-responders with a cold microenvironment and low SQSTM1 expression) observed with PD-L1 expression assessment alone.

FIGS. 10 to 16: SQSTM1 depletion is sufficient to induce a “COLD” phenotype and a DDA resistance.

FIG. 10 represents Heat map (up) and corresponding GSEA profiles showing significant enrichment of gene sets associated with the ‘Hot’ phenotype [CD274, antigen presentation (bottom left)], Cisplatin/RT sensitivity (RT Radiotherapy), and DNA repair (bottom, left, right) across 190 lung cancer cell lines with high and low SQSTM1 expression, respectively. The blue and red colors correspond to low and high expressions, respectively.

FIG. 11 represents Western blot showing an efficient decrease in SQSTM1 protein levels after shRNA knockdown with two independent SQSTM1 shRNA (SQSTM1 #1, and #2 vs. Control shRNA). A549 lung cancer cell responses to SQSTM1 depletion were then examined with regard to HLA-B and DNA repair (RAD51, and P-Thr68-CHK2 WB);

FIG. 12 represents chemokine expression (CXCL10, IL29) after shRNA knockdown. Gene expression in A549 expressing control or SQSTM1 shRNAs, as well as shSQSTM1 cells transfected with SQSTM1 plasmid (for rescue, #2+SQ), was measured by qRT-qPCR. Similar results were observed in 3 independent experiments.

FIG. 13 represents MHC-I expression (HLA-A-C, qRT-PCR) after shRNA knockdown. Gene expression in A549 expressing control or SQSTM1 shRNAs, as well as shSQSTM1 cells transfected with SQSTM1 plasmid (for rescue, #2+SQ), was measured by qRT-qPCR. Similar results were observed in 3 independent experiments.

FIG. 14 represents A549 cell viability (Cisplatin dose-response, IC50, Cis: Cisplatin 5 days) after SQSTM1 shRNA knockdown.

FIG. 15 represents HLA-B (left) and PD-L1 (right) gene expression in response to DDA treatment (Cis: Cisplatin, 10 μM, Oxa: Oxaliplatin, 1.4 μM, Dox: Doxorubicin, 50 nM, RT: Ionizing Radiations, 10Gy, five days).

FIG. 16 represents MHC-I (A-C, up, flow cytometry) and PD-L1 cell-surface expression (bottom, flow cytometry) in A549 transduced with control, SQSTM1, or ATG5 shRNA. Similar results were observed in 3 independent experiments. The maximum enhancement of HLA-B and PD-L1 expression was achieved by Cisplatin, which was chosen for other experiments.

FIG. 17 represents the consequence of SQSTM1 depletion on DDA resistance. IC50 concentration from Crystal Violet Cytotoxicity Assay (right) in shControl and shSQSTM1 A549 cells after five days of treatment with the indicated DDA (N=3).

FIGS. 18 to 22: DNA damage agents induce late expression of PD-L1/MHC-I in an SQSTM1-dependent manner.

FIG. 18: shControl and shSQSTM1 A549 cells were treated with 10 μM of Cisplatin. At the indicated time, global DNA damage (A) was measured by 53BP1 focus formation (right immunofluorescence staining, 53BP1 red, Dapi, blue. Left, cell percentage with more than five spots).

FIG. 19: SQSTM1-dependent activation of TBK1 and JAK pathways in cisplatin-treated A549 cells. Cells were lysed, and activation of TBK1 and JAK pathways was assessed by Western blotting of WCL with anti-phospho-Ser172-TBK1 (P-TBK1) and anti-phospho-Tyr701-STAT1 (P STAT1). Tubulin and TBK1 were used as loading controls (N=3).

FIG. 20: Relative expression of type I and III Interferons (left, five days-5d, N=3), and kinetic of IL29 expression were measured by qRT-PCR (right, N=3).

FIG. 21: Time course of Cisplatin-induced HLA-B mRNA (left, qRT-PCR, N=3), protein (middle, top: western blot, bottom: Fold change for densitometry by ImageJ normalized to tubulin, N=4), and cell surface expression (flow cytometry, right, N=3). Median Fluorescence Intensity (MFI) and % of positive cells are indicated.

FIG. 22: Time course of Cisplatin-induced PD-L1 mRNA (left, qRT-PCR, N=3), protein (middle, top: western blot, bottom: Fold change for densitometry by ImageJ normalized to tubulin, N=4), and cell surface expression (flow cytometry, right, N=3). Median Fluorescence Intensity (MFI) and % of positive cells are indicated.

FIGS. 23 to 24: Cisplatin induces cell cycle arrest, and DNA methylation independently of SQSTM1.

FIG. 23: CDKN1A, and DNMT1 gene expressions in cisplatin-treated shControl and shSQSTM1 A549 cells (10 μM, qRT-PCR; N=3). FIG. 24] FIG. 24: GSEA and box-plots analyses correlating the ICI response with DNA repair signatures, cell cycle, and methylation, DNA (anti-PD-1, cBioportal, https://portals.broadinstitute.org/singlecell/study/melanoma-immunotherapy-resistance #study-visualize).

FIG. 25: Immunogenic cell death (ICD) inducers up-regulate HLA-B and PD-L1 expression through a SQSTM1-dependent pathway.

FIG. 26: shSQSTM1 A549 cells were treated with ICD inducers (Radiotherapy, 10Gy; Doxo, 50 nM). qRT-PCR expression of DNMT1, IFNL2/IL29, PD-L1, and HLA-B in DDA-treated shControl and shSQSTM1 A549 (time course; N=3).

FIGS. 26: Cisplatin induces HLA-B and PD-L1 expression in TBK1 and JAK-dependent manner

FIG. 26: shControl A549 cells were treated with 10 μM of Cisplatin in the presence or absence of the TBK1 inhibitor MRT 67307 (10 μM) or JAK1/JAK2 inhibitor Ruxolitinib (5 μM). Left, phospho-TBK1, and phospho-STAT1 western blot (5 days). Right, qRT-PCR expression of IL-29, HLA-B, and PD-L1 (100% corresponding to the Cisplatin treated cells (right panel; N=2).

FIGS. 27 to 28: IFN rescues the upregulation of HLA-B and PD-L1 expression in SQSTM1-depleted cells.

FIG. 27 represents that autophagic defect increases the IFN sensibility. SQSTM1, ATG5, or ATG7 knockdown A549 cells were treated by IFNG (50 ng/ml) 1 h or 24 h. Western blot of Phospho-STAT1 and IDO1 (Actin was used as a loading control). PD-L1 expression was analyzed by qRT-PCR and cell surface staining of PD-L1 expression (right).

FIG. 28 represents phospho-TBK1, phospho-STAT1, HLA-B and PD-L1 western blots after Cisplatin (5 d), and IFN (24 h) (TBK1 was used as loading control; right panel; N=3).

*<P 0.05 Nonparametric T-test (Mann-Whitney)

FIGS. 29 to 30: Docetaxel induces HLA-B and PD-L1 expression in SQSTM1-depleted cells.

FIG. 29 represents the relative expression of CDKN1A/p21, DNMT1, IFN-III, HLA-B, and PD-L1 in docetaxel-treated shControl and shSQSTM1 A549 cells (5 nM, qRT-PCR, N=2).

FIG. 30 represents Phospho-TBK1, phospho-STAT1, and HLA-B western blots of Cisplatin-, and docetaxel treated cells (5 nM, 5 days, Actin was used as a loading control).

FIG. 31 represents representative images of SQSTM1 stainings (original magnification×400). The skin melanoma shows increased cytoplasmic and nuclear SQSTM1 staining patterns.

### EXAMPLES

### Example 1

After the recent advent of Immune Checkpoint Inhibitors (ICIs), the challenge of clinical cancer trials is to develop the optimal combinations of ICIs with DNA-Damaging Agents (chemotherapy and radiotherapy, named DDAs hereafter). Elucidating resistance mechanisms is essential to propose new predictive biomarkers and new therapeutic approaches to improve ICI efficiency. The inventors hypothesized that resistance to DDA and ICIs is mediated in part by intrinsic tumor mechanisms, some of which may be shared. Through three complementary approaches (in silico, ex vivo on patient cohorts, and in vitro), the inventors identify the p62/SQSTM1 scaffold protein as a key molecular mediator capable of predicting and controlling sensitivity DDA and ICIs. Mechanistically, in response to DNA damage, the inventors found that SQSTM1 is essential for the inhibition of DNA repair. Treating the SQSTM1-depleted tumor cells with docetaxel can rescue the IFN, and MHC pathway, providing a promising therapeutic avenue turning a cold into a hot tumor in non-responders. Depending on its levels, the inventors thus propose SQSTM1 as a predictive biomarker for guiding treatment decisions between ICIs alone, ii) ICI combined with Cisplatin, iii) or ICI combined with docetaxel, aimed at increasing ICI efficacy and patient outcomes.

### Results

The Inventors compared RNA expression signatures from cohorts of patients treated with radiotherapy, chemotherapy, and immunotherapy to identify shared molecular pathways that may mediate cross-resistance. The inventors classified tumors into immune “hot” and “cold” based on the expression of cytotoxic T lymphocytes (CTL) markers CD8A and CD8B, the immune checkpoint gene CD274/PD-L1 (hereafter referred to as PD-L1), and the Class-I MHC genes (HLA-A, B, C). The enrichment in CD8+ T cells was further validated by the presence of two cytolytic enzymes, granzyme B (GZM B) and perforin (PRF).

### 1) Evidence of a Cross-Resistance Between DDA and ICIs Strategies

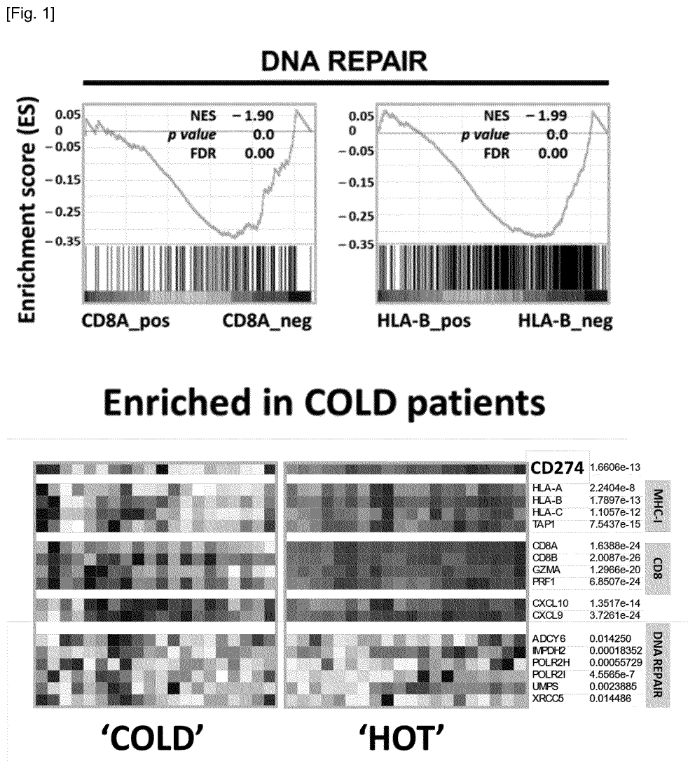

Gene set enrichment analysis (GSEA) of two malignancies eligible for immunotherapy, lung adenocarcinoma, and melanoma highlights that cases, which lacked tumor-infiltrating lymphocytes (TILs) and expressed lower levels of MHC-I, two features of cold tumors, overexpressed the DNA repair markers (FIG. 1). Likewise, a pan-cancer analysis supported the association of DNA repair signature with “cold” tumor phenotype across diverse tumor types (FDR<0.01, data not shown). Notably, this higher DNA repair signature was a consistent hallmark of the majority of cold tumors. As a significant source of genomic stability, higher DNA repair-tumors tend to be correlated to lower tumor mutation burden (TMB), as it was for low antigenicity and lack a T cell-inflamed tumor microenvironment, three predictive biomarkers of the poor clinical benefit of ICIs. Given such accumulating evidence, the inventors propose that i) higher DNA repair may present another opportunity to optimally select patients for ICIs therapy. So far, very small fractions of cancers occurring from DNA repair defects were found responsive to ICI therapy. It now appears that a clinically significant fraction of cancers has DNA repair defects. ii) Also, by inference, it might be expected that the immunosuppressed, cold tumors are intrinsically coupled to DNA repair and thereby associated with inferior responses to DDA than the “hot” tumors.

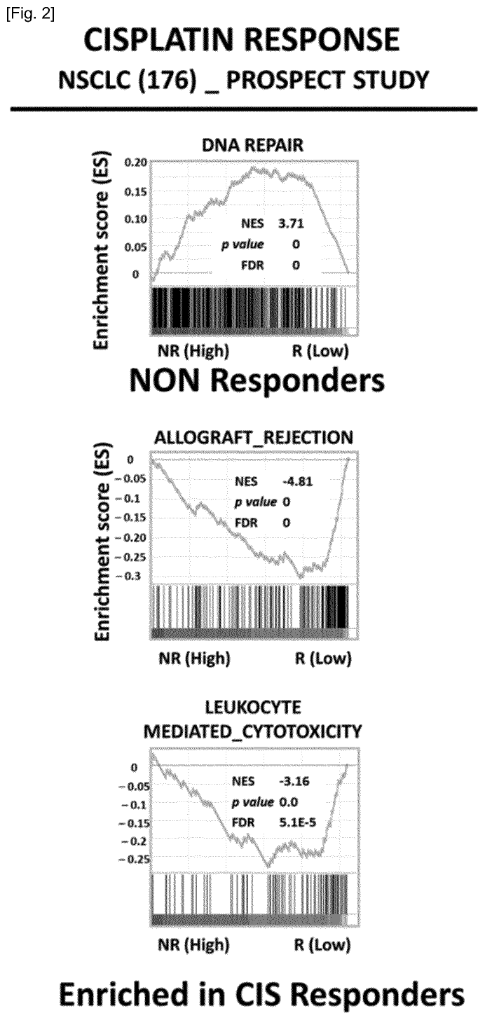

The inventors therefore analyze whether DNA repair coupled with hot/cold signatures could be applied to predict the clinical responses to the three therapeutic options: Chemotherapy, radiotherapy, and immunotherapy. Instead, hierarchical clustering based on gene expression revealed the enrichment of hot signature (T-cell markers and MHC-I) within Cisplatin-sensitive, and radiotherapy (RT)-sensitive cancers, three hallmarks that determine the immunotherapy responses both in LUAD (FIG. 2) and independent validation cohorts of other cancer types (Data not shown). Conversely, an immune defective ‘cold’ signature is a shared feature of DDA-resistant tumors, highly suggesting that the antitumor immunity is critical for the clinical activity of Cisplatin/RT. Intriguingly, the inventors provide the first evidence of a significant correlation of DNA repair signature with ICI resistance (FIG. 3). By inference, such recurrent clinical correlations offer the rationale for combining DDA with ICIs to improve the outcome of ICIs/DDA sensitive patients. Along this line, a subgroup of patients refractory to Cisplatin/RT would also acquire cross-resistance, decreasing the benefit from second-line immunotherapies. An attractive possibility to explain this aggressive resistant tumor phenotype might be to consider that a high DNA repair pathway, known to mediate resistance to conventional DDA, might also contribute to a diminished response to immunotherapy.

### 2) SQSTM1/p62 is a Promising Biomarker of ICIs/DDA Responsiveness

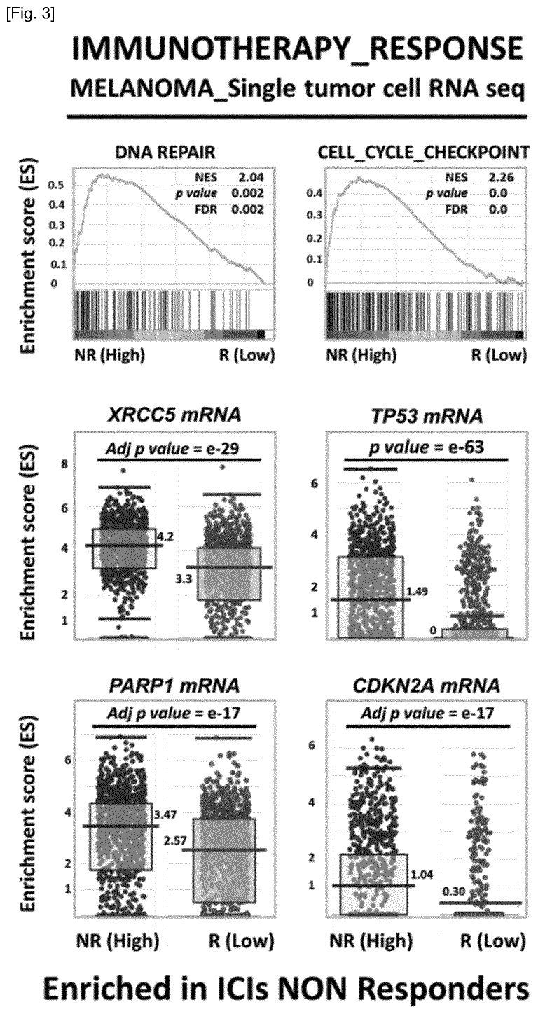

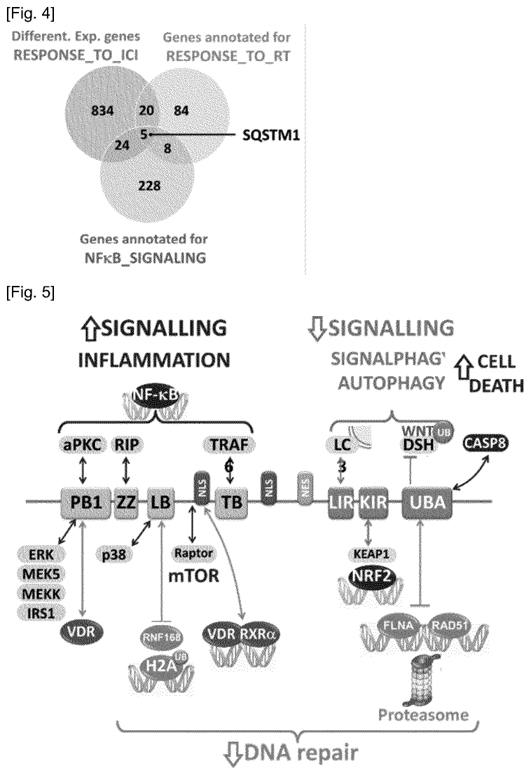

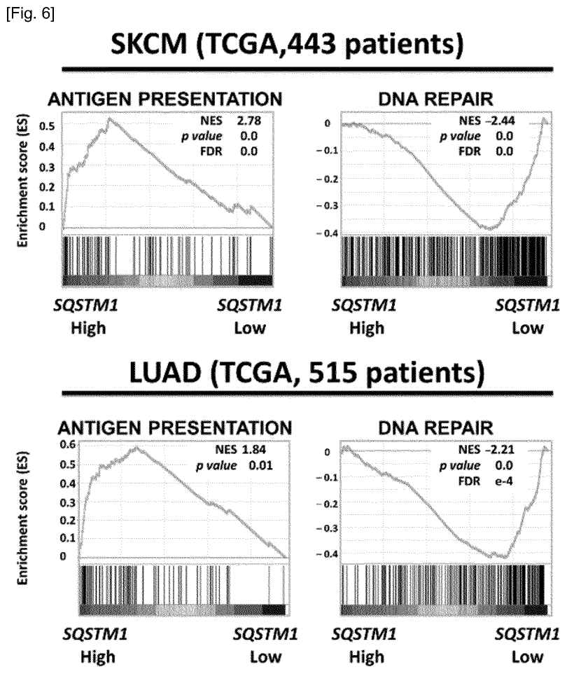

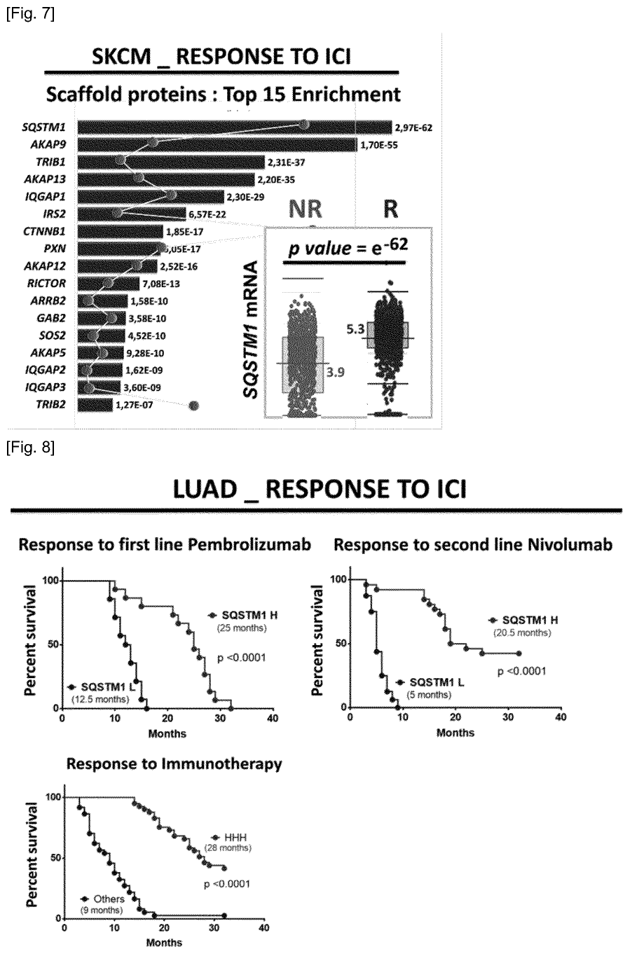

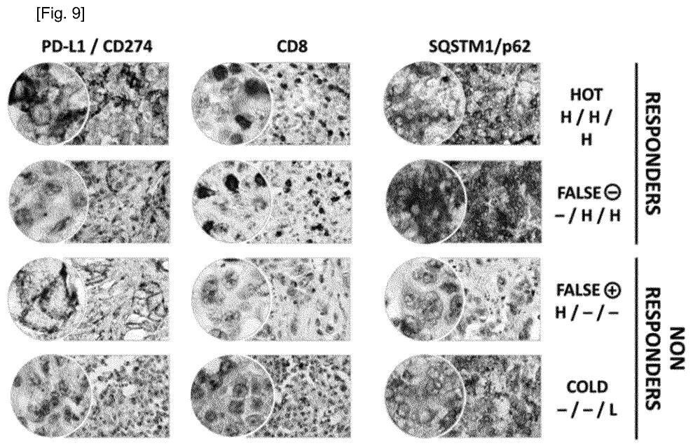

The co-expression of cold and DNA repair signatures suggests that the responses to ICIs and DDA are not only tightly coupled to promote tumor cross-resistance but also likely controlled by the same signaling pathway. The inventors' strategy was then to identify in silico a signaling scaffold that was differentially expressed between the cold/hot phenotype and, most importantly, correlated to the responsiveness to the combination ICI/DDA (FIG. 4). From the potential signaling platforms screened, the inventors decided to focus their attention on the scaffold protein SQSTM1 because i) it helps integrate and relay the signals from the cell surface to the nucleus by docking several signaling partners of inflammation, DNA repair, and cell death pathways (FIG. 5). ii) Interestingly, its expression was positively correlated with antigen presentation and inversely with DNA repair signatures (p=e-9) in lung adenocarcinoma and melanoma (FIG. 6). iii) SQSTM1 is the most highly enriched platform in DDA sensitive-tumors, whereas it is not in resistant-tumors (Data not shown). iv) The inventors provide the first evidence of SQSTM1 overexpression in ICIs responders in comparison with non-responders using single-cell RNA-seq of melanoma (p=e-62) (FIG. 7). This was validated by using two independent cohorts of advanced LUAD patients (stages IIIA-IV, in progress) treated in first-line with blocking PD-1 (Pembrolizumab) or in second-line with PD-1 (Nivolumab) immunotherapies (FIG. 8), in which a high detection of SQSTM1/p62 by IHC was again associated with ICIs responsiveness (p<0.0001). Strikingly, low SQSTM1 expression was sufficient to identify a subset of PD-L1 “false-positive” tumors (8 patients, 10.4%) that showed a low CD8 T lymphocyte infiltration and failed to respond to immunotherapies (FIG. 9). Overall, the inventors' results suggest the clinical utility of tumor SQSTM1 in predicting responsiveness to ICIs, DDA as monotherapy, and potentially ICIs+DDA Combi (in progress).

### 3) SQSTM1 is Essential for DDA-Induced Toxicity and Enhanced Antigen Presentation

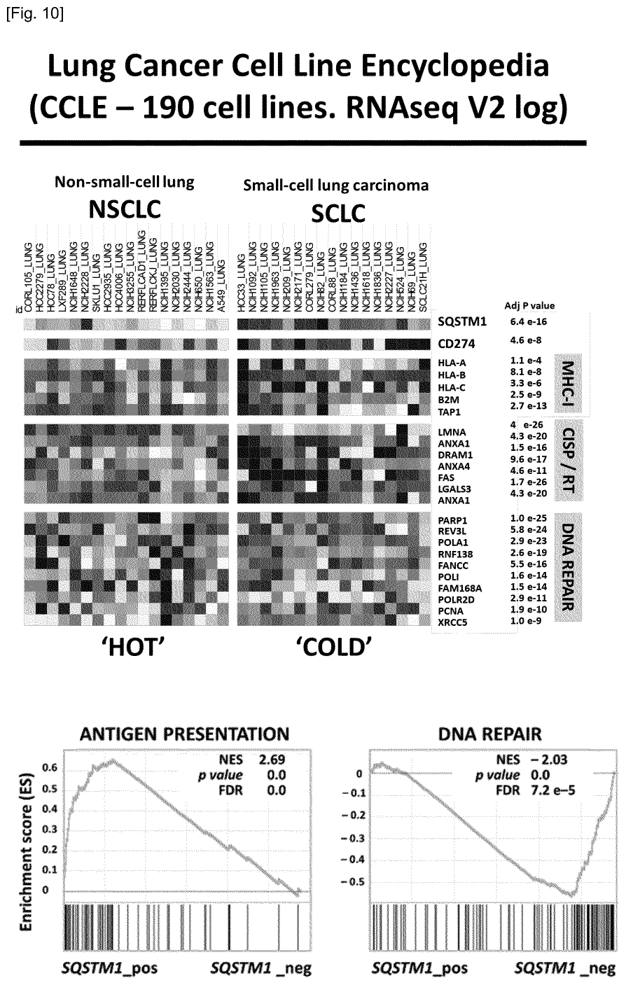

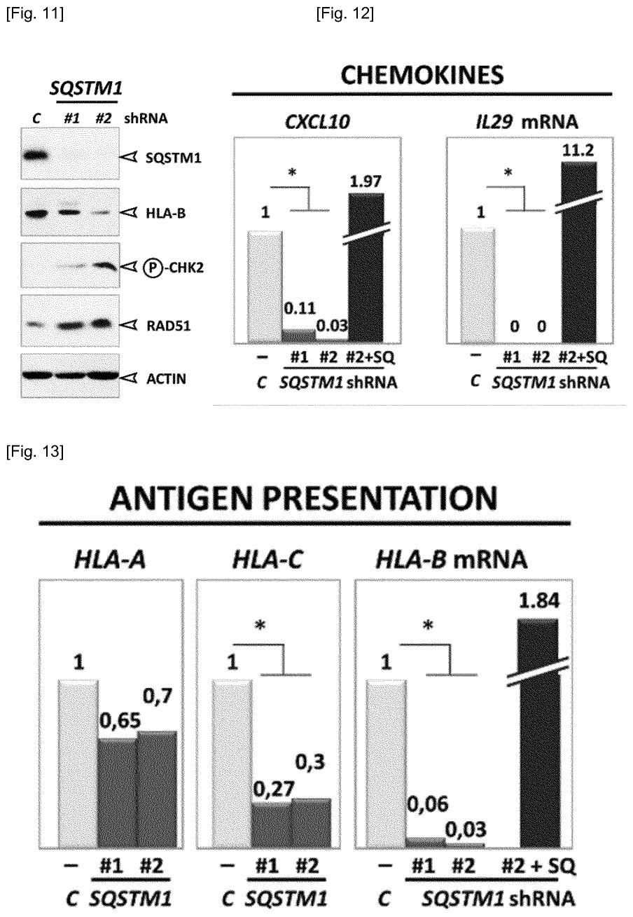

Given the above clinical results, the inventors' working hypothesis is that the expression of SQSTM1/p62 might dictate the cross-response to ICIs and DDA. Using a panel of 190 lung cancer cell lines from CCLE, the inventors first confirmed by GSEA that the tumor-cell-intrinsic expression of SQSTM1 is positively correlated with antigen presentation, and inversely with DNA damage repair and DDA/ICIs resistance gene signatures (FIG. 10). Along this line, it is worth pointing out that the expression of SQSTM1 is lowest in the small cell lung cancer cell lines (SCLC, p-value=6 e-16), one of the most aggressive cancer types that is resistant to ICIs and DDA (FIG. 10 right). The inventors then assessed the causal relationship between SQSTM1 and cross-sensitivity to anti-cancer therapies. As a cellular model, the inventors chose the A549 cell line, derived from a lung adenocarcinoma bearing the KRAS G12S oncogene, and the loss of the SKT11/LKB1 tumor suppressor gene (Q37*), two molecular events associated with a primary ICIs resistance, while being sensitive to chemotherapy. Experimentally, SQSTM1 knockdown in A549 cells by shRNA (FIG. 11) was sufficient to recapitulate the severe phenotype observed in silico faithfully; as evidenced by the down-regulation of the chemokine CXCL10, the IFN-III IL29 (FIG. 12), and the MHC-I (HLA-A, B, and C FIG. 13) both at the mRNA level and HLA-B protein level (FIG. 11). In keeping with this scenario, the re-expression of SQSTM1 restored the expression of chemokines and HLA-B (FIGS. 12 and 13). In agreement with previous observations, silencing of SQSTM1 was also sufficient to increase the expression of DNA repair marker RAD51, and the phosphorylation of the checkpoint kinase 2 (CHK2), which delays cell cycle progression to facilitate DNA repair (FIG. 11).

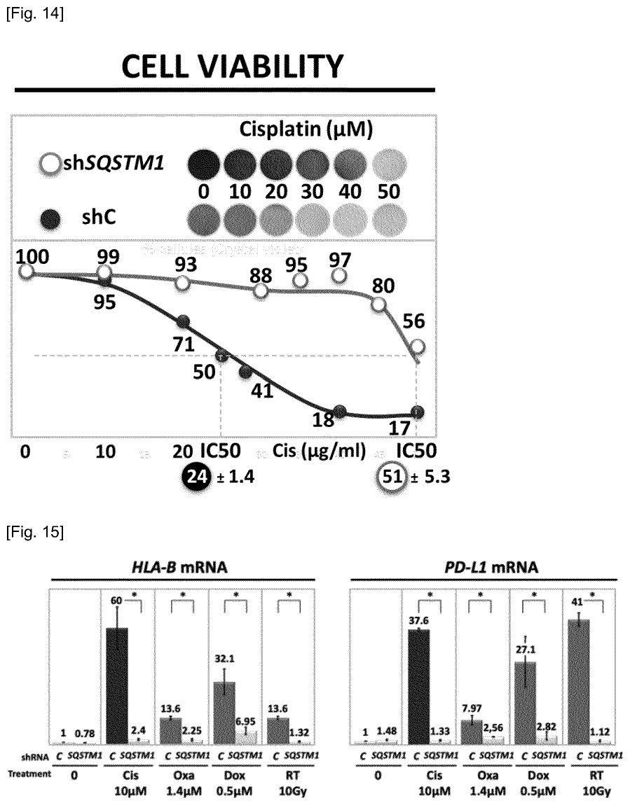

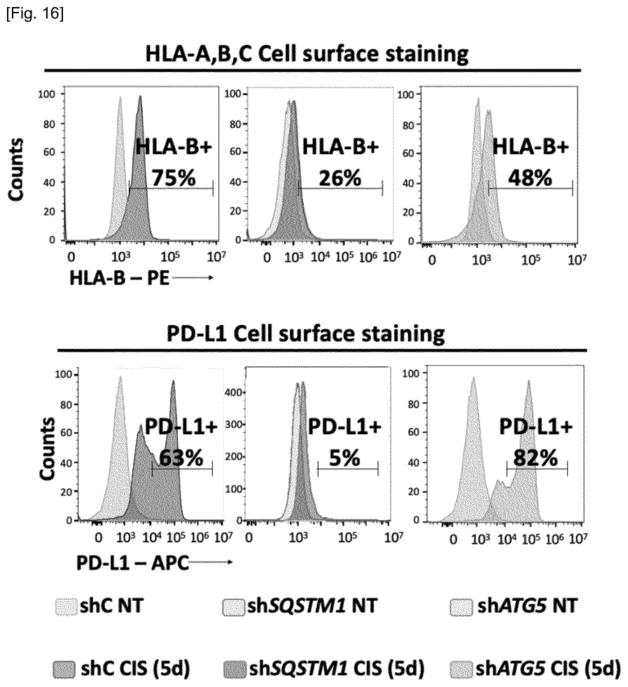

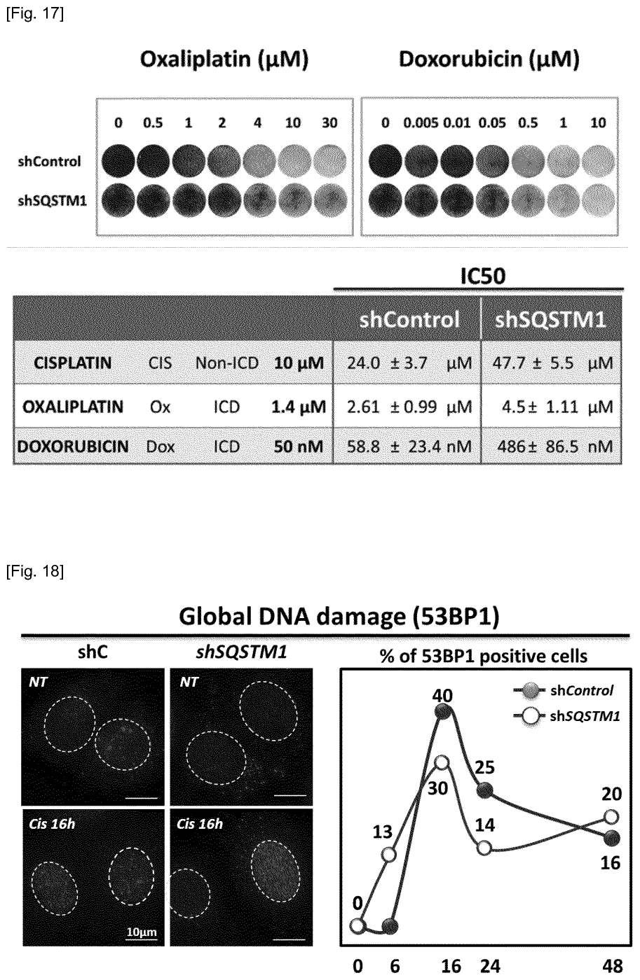

Therefore, the inventors explore the sensitivity of the SQSTM1-depleted cells to many FDA-approved chemotherapeutics in clinical trials with ICI. Upon Platinum treatment (Cisplatin, Cis), ShSQSTM1 cells were consistently resistant (with an IC50 51±5.3 μM twice that of control ShC cells), compared to the massive death of control ShC cells (IC50 24±1.4 μM, FIG. 14). At sublethal doses (10 μM), the inventors observed that Cis induced in ShC cells a robust overexpression of HLA-B and PD-L1 mRNA (60-fold and 37.8-fold increase, respectively; FIG. 15) reflected by the enhanced presence of the MHC-I (HLA-A, B, C) and PD-L1 at the cell surface (FIG. 16). In total agreement with Cisplatin's resistance, the depletion of SQSTM1 canceled the Cis-upregulated HLA-B and PD-L1 expressions (FIGS. 15 and 16). As a proof-of-concept, all these features (i.e., DDA resistance and decreased HLA/PD-L1 expressions) were recapitulated using different DNA damaging agents, such as anthracyclines (Doxorubicin, Dox, 50 nM), oxaliplatin (Oxa, 1.4 μM), and radiotherapy (RT, 10Gy) (FIGS. 15 and 17).

Considering that SQSTM1 functions both as a scaffold protein and a selective autophagy receptor; the inventors found that inhibiting autophagy by ATG5 shRNA (FIG. 16) or pharmacological bafA1 or chloroquine treatment (data not shown) did not recapitulate the phenotype induced by SQSTM1 deficiency but instead led to enhanced expression of HLA-B and PD-L1. Together, these results point to the notion that the resistances to cancer therapies are, at least in part, an intrinsic property of tumor cells. At the molecular level, these results establish that the SQSTM1 signaling scaffold rather than autophagy pathway is critical for DDA-induced toxicity, HLA-B, and PD-L1 expressions, the key immune checkpoint protein targeted in immunotherapy. Collectively, this unveiled an additional function of SQSTM1 in ICI/DDA cross responses.

### 4) SQSTM1 is Absolutely Required for the Late Up-Regulation of IFN/PD-L1/MHC-I by Cis.

The inventors next assessed how the loss of SQSTM1 impacted the DDA-induced responses. Besides a direct cytotoxic effect, It has been proposed that DDA's therapeutic action may depend on the release into the cytosol of DNA, which being recognized as a DNA virus, primes an early IFN response (IFN, HLA-B, and PD-L1) at 16 h.

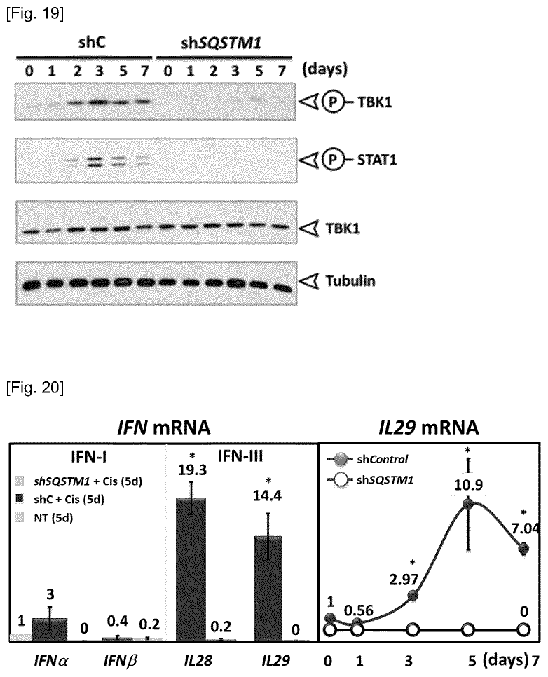

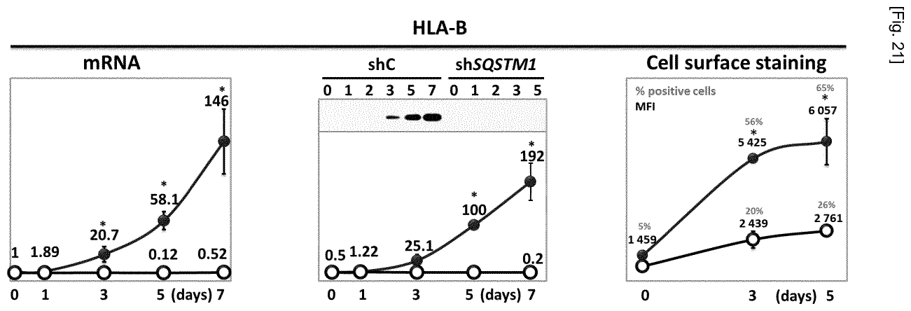

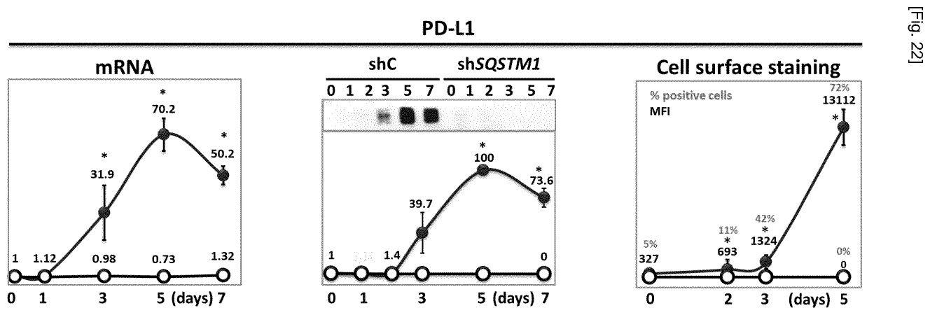

As expected, DDAs rapidly induced DNA damages, as visualized by the phosphorylation of CHK2 (data not shown) and the formation of the 53BP1-positive foci (FIG. 18), which started after 6 hrs of Cisplatin treatment, reached a peak value similarly at 16 hrs, then declining. Unexpectedly, in DDA-treated ShC cells, phosphorylations of TBK1 and STAT1 (FIG. 19), as well as the expression of IFN-I and IFN-III, did not coincide with that of DNA damage but started later around the 3rd day of treatment (FIG. 20). Downstream, the kinetics of DDA-induced HLA-B and PD-L1 mRNA (right) and protein (middle and left) expression followed that of the IFNs, reaching the plateau value around the 5th day (FIGS. 21 and 22). Here again, the DDA upregulation of phospho-TBK1, IFNs, HLA-B, and PD-L1 expressions failed in the absence of SQSTM1 throughout the time course (FIGS. 19 to 22). This observation prompted us to identify the molecular pathway controlled by SQSTM1 between DNA damage and the immunogenic response.

Such kinetic discrepancy argues against a major role of the early dsDNA in the initiation of the late IFNs/HLA-B/PD-L1 pathway. This in line with the silencing of the DNA sensor STING in LKB1/STK11 mutated A549 cells and instead suggested an alternative SQSTM1-dependent DAMP.

### 5) SQSTM1 and DNA Damaging Agents.

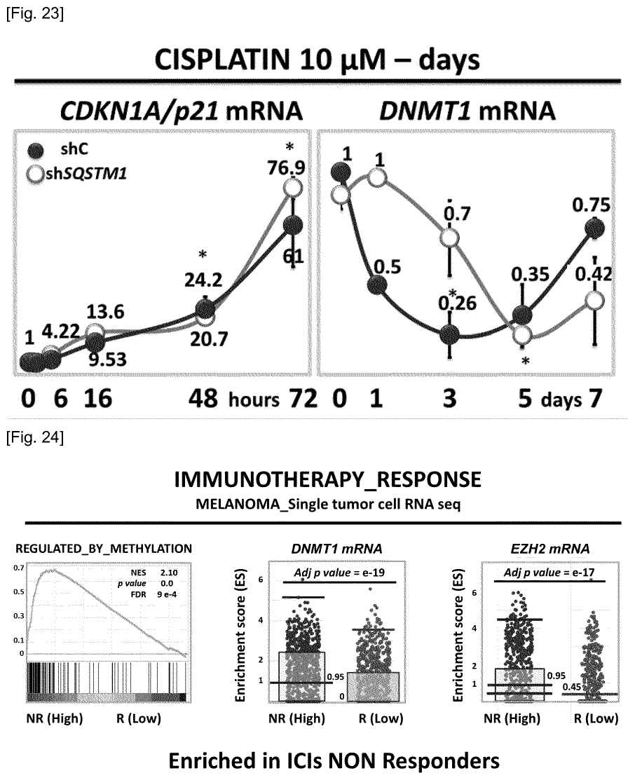

Remarkably, the inventors observed that the early induction of cell cycle inhibitor CDKN1A/p21 faithfully tracked that of DDA-induced DNA damages (nuclear foci, FIG. 18) in both control and SQSTM1-depleted cells (FIG. 23). Together, this identifies that SQSTM1 is dispensable for the early first steps of DNA damage response and acts after the formation of DSBs and the cell cycle arrest.

Recently, scarce preclinical studies have evidenced that drugs inducing cell cycle arrest and DNA demethylation guide IFN/MHC-I/PD-L1 expression in cancer cells. In support of this hypothesis, it turned out from in-silico analysis that the ICI expression was inversely correlated with the expressions of DNA methylases (FIG. 24). Of a panel of epigenetic regulators (DNMT1, DNMT3a, DNMT3b, LDS1, SETD1, TRIM28/KAP1, EZH2) reported to up-regulate PD-L1 expression, only the expression of DNMT1 fell early at 1d of Cis treatment, while the others showed modest or variable responses (FIG. 23). In support of DDA-mediated DNMT downregulation as the initiating events, the treatment of A549 ShC cells with 5-Azacytidine (500 nM, 5-Aza), a demethylating agent that inhibits DNMT1, recapitulated the late expression of HLA-B and PD-L1 (at 5 and 6 days). This event was consistently preceded by the reactivation of IFN-III IL28 (5 days)

Notably, in the absence of SQSTM1, we observed a similar downregulation of CDKN1A/p21 and DNMT1 expression upon Cisplatin treatment (FIG. 23). But, this was not followed by the activation of the entire downstream pathway from P-TBK1/IFN/p-STAT1 (FIG. 19). Consistent with their aberrant signaling, SQSTM1-depleted cells did not up-regulate the mRNA, protein, and cell surface expression of HLA-B and PD-L1 in response to Cisplatin.

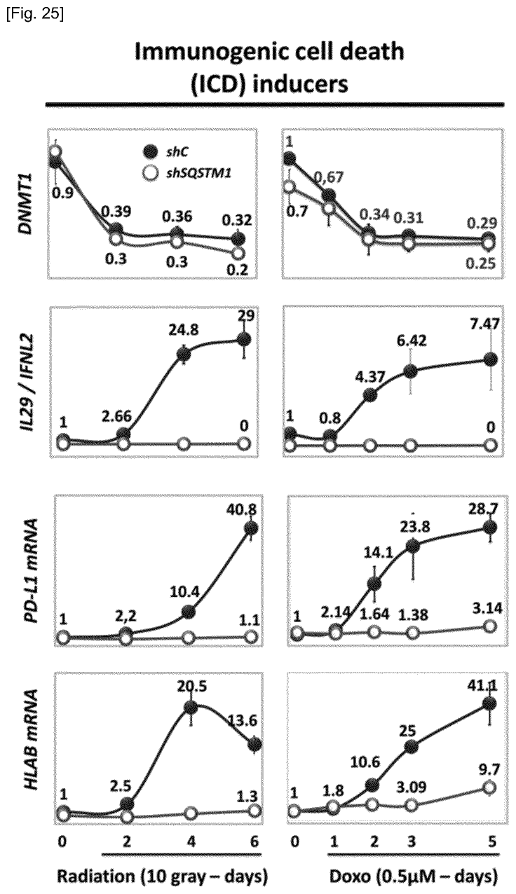

Beyond Cisplatin, the inventors then recapitulated the late induction of this pathway with different immunogenic cell death (ICD) inducers such as Radiotherapy, Oxaliplatin, and Doxorubicin. Regardless of ICD inducers, they showed that this pathway was entirely dependent on SQSTM1 (FIG. 25). These data therefore reveal a novel and global role of SQSTM1 on the DNA damaging agent-induced PD-L1/MHC-I expression.

### 6) DDA Induce and HLA-B and PD-L1 Expression Through Activation of the TBK1-IFN-JAK Pathway

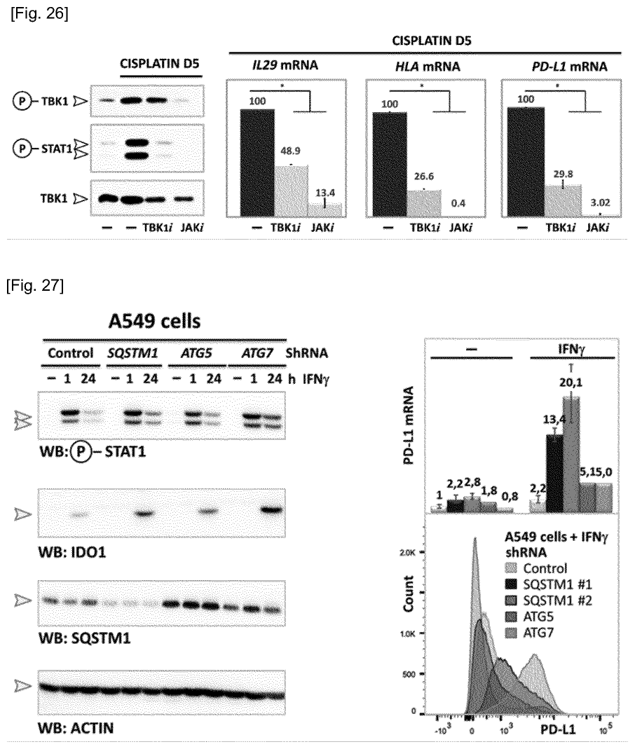

The inventors show that Cisplatin-induced the phosphorylation of TBK1, a kinase involved in the transcription of IFN. Consistently, they detect IFN-III at the mRNA levels, and co-treatment of Cis with TBK1 inhibitor MRT 67307 was sufficient to block Cisplatin ability to induce IFN/HLA B/PD-L1, as did the JAK/STAT inhibitor ruxolitinib (FIG. 26). Altogether, inventors' data suggest that sublethal dose DDA can induce the expression of type III, but not type I, IFNs, followed by a downstream expression of HLA-B and PD-L1 in a JAK-dependent manner.

### 7) IFN Rescue the Upregulation of HLA-B and PD-L1 Expression in SQSTM1 Depleted Cells.

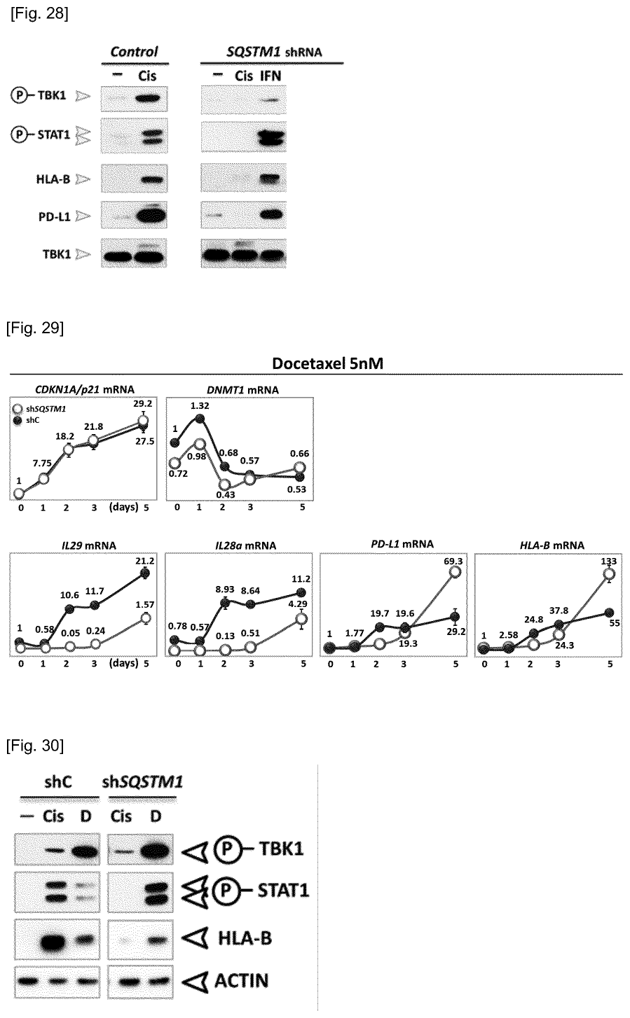

The inventors then checked that the addition of IFNG induced STAT1 phosphorylation, HLA-B, and PD-L1 expression in shC and shSQSTM1 cells, indicating that the IFN pathway was functional in SQSTM1-depleted cells (FIGS. 27 and 28). Of interest, we observed that the expression of the ISG (IDO-1, HLA-B, and PD-L1) in response to IFNG was higher for SQSTM1-depleted cells compared to Shc. This pattern was recapitulated, at least in part, in IFN-stimulated shATG5 and ShATG7, suggesting that autophagy-deficient cells exhibit greater IFN sensitivity (FIG. 27).

Together, these data highly suggest a working model in which DDA drives the sequential pathway from DNA damage, G1 arrest, toward DNMT downregulation, and downstream IFN controlled MHC and PD-L1 expression. In this pathway, we conclude that SQSTM1 controls the production of MHC/PD-L1 downstream of demethylation.

### 8) Taxane Overcomes the Expression of HLA-B and PD-L1 in SQSTM1-Depleted Cells

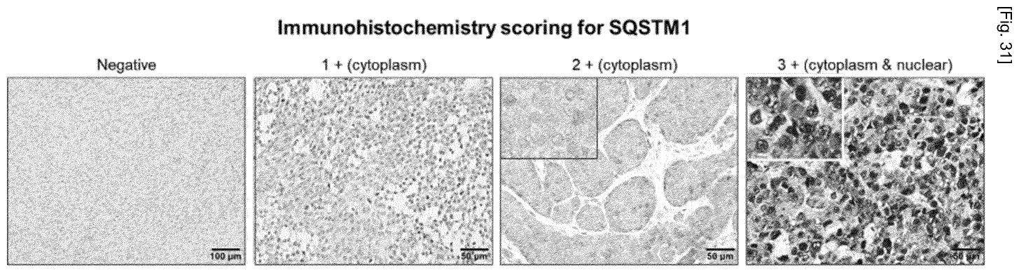

At that stage, it was of interest to identify a standard-of-care treatment that could overcome the resistance of SQSTM1-depleted cells. Of, microtubule targeting agents (MTA) are candidates that are second-line chemotherapies with proven efficacy for DDA-resistant cancers. We show that docetaxel did trigger growth arrest and DNMT1 downregulation in both Shc and ShSQSTM1 cells. Surprisingly, despite the absence of SQSTM1, docetaxel significantly rescued the downstream TBK1/STAT1 phosphorylation (FIG. 30) and the expression of IFN, HLA-B, and PD-L1 (FIGS. 29 and 30). Notably, docetaxel followed the same late time course observed for other DDA. Here again, it is worth pointing out that Cisplatin remained the most effective drug for inducing HLA-B and PD-L1 in SQSTM1 positive cells, while docetaxel was the sole effective in SQSTM1-depleted cells. Altogether, our data uncovered a new property of docetaxel in inducing HLA-B and PD-L1, providing the rationale for exploring a novel combination with ICI, especially in DDA-resistant patients. Of particular interest, SQSTM1 emerges to be a powerful biomarker that may not only predict ICI/DDA responses but also may guide treatment decisions between two ICI combinations with Cisplatin or Docetaxel.

### DISCUSSION

Lung cancer is the leading cause of cancer-related deaths, more than the skin, colon, prostate, and pancreas cancers combined. DNA damaging agents, such as Platinum-based chemotherapeutics and ionizing radiation, are standard-of-care treatments, and about 80% of lung cancer patients will receive these therapies during their course of treatment. However, despite the initial response, almost all patients eventually develop DDA-resistant relapse, engaging patient survival. Nowadays, immunotherapy with anti-PD-1 or anti-PD-L1 neutralizing antibodies shows therapeutic promises for advanced patients. However, despite this significant advance, 30-40% of patients still demonstrated resistance to immunotherapy.

The limited success of sequential combination ICIs+DDA therapy remains elusive and likely results from the narrow sensitive therapeutic window. Moreover, the landscape of resistance to ICIs/DDA is unknown, with no biomarker capable of accurately segregating responders from non-responders. So far, tumor expression of PD-L1 is associated with enhanced objective response rates to PD-1/PD-L1 inhibition but is neither sensitive, specific, nor useful for predicting the combination response. Although critical for therapeutic interventions, how DDAs up-regulate antigen presentation and PD-L1 is not entirely clear, nor do we appreciate if this pathway is inhibited in DDA-resistant tumors.

The inventors' data led to the provocative proposal that the scaffold protein SQSTM1 may positively predict clinical outcomes of ICIs, DDA, and potentially ICIs/DDA combinations. This hypothesis is based on three key observations the inventors have made, both in silico, in vivo using patient cohorts, and in vitro using engineered silenced cell-lines: i) SQSTM1 mRNA and protein expressions (IHC scores) are significantly higher in ICIs, RT, and Cis responders than in non-responders. ii) Mechanistically, SQSTM1 controls both the expression of DNA repair and immune IFN/MHC/PD-L1 pathway. iii) SQSTM1 loss is sufficient to drive innate resistance to ICIs and DDA therapies. The inventors aim now to evaluate the effect of genetic and pharmacologic inhibition of SQSTM1 (zz inhibitor, CRISP/CAS9) on the tumor immune microenvironment, particularly T cell-mediated anti-tumor immunity (T cell infiltration and activation) using co-culture assays and syngeneic in vivo murine NSCLC model.

### Sqstm1 is a Molecular Driver of Immune Tumor Plasticity

Here, the inventors provide the first evidence that SQSTM1 expression defines subgroups of LUAD and SKCM with distinct biology, immune profiles, and therapeutic vulnerabilities. Depending on its level, SQSTM1 drives two completely different immunosuppressive programs: The tumors with low SQSTM1 level were indeed associated with a “cold” microenvironment, with poor antigen presentation and T cell exclusion, while those with high SQSTM1 expression were “hot” with PD-L1 expression, T cell infiltration, and exhaustion.

Of interest, SQSTM1 is a critical scaffold protein involved in the activation of key signaling pathways that control inflammation, cell survival (NF-κB), oxidative detoxifying stress (NRF2), and cell growth (mTOR); all program events that have a direct impact on cancer development. As such, the oncoprotein SQSTM1 couples tumor growth with immune evasion. Not surprisingly, SQSTM1 is essential for KRAS-G12D-induced lung tumorigenesis in mice. In humans, SQSTM1 overexpression was associated with worse survival in lung, gastrointestinal, prostate, liver, kidney, and breast cancers. Paradoxically, SQSTM1 loss was also shown to increase prostate cancer tumorigenesis, a cold cancer type. In this regard, the ability of SQSTM1 gain and loss the inventors unveiled herein to conferring tumor immune evasion may help explain these controversial results.

Besides these signaling functions, SQSTM1 was the first identified autophagic receptor, a cellular process that promotes tumor cell survival and drug resistance. Autophagy, therefore, mediates the clearance of SQSTM1, and inhibiting autophagy by ATG5 shRNA resulted in SQSTM1 accumulation and consistently in HLA-B overexpression. Together, these data highly suggest a working model in which the remarkable control of SQSTM1 expression by feedback loops involving oncogenes, inflammatory cytokines, and autophagy degradation might dictate the tumor cell plasticity between the “HOT” and “COLD” phenotypes.

### Sqstm1 Governs DDA and ICIs Sensitivity Via Inhibition of DNA Repair

The inventors show here that SQSTM1 downregulation is a major driver of resistance to DDA and ICIs. Mechanistically, SQSTM1 repressed DNA repair and concomitantly was essential for the expression of MHC-I. SQSTM1 represents, therefore, a molecular link between DDA sensitivity, tumor DNA instability, tumor mutation burden, and tumor immunity. Further investigation of potential mechanism, the role of SQSTM1 within the nucleus is still not well understood. SQSTM1 contains two nuclear localization signals and one nuclear export signal, which allow SQSTM1 to shuttle continuously between nuclear and cytosolic compartments at a high rate. Upon DNA damage, the inventors found that SQSTM1 was recruited to nuclear DNA damage foci (data not shown), where it was reported to inhibit DNA repair. Besides DNA repair, SQSTM1 was also reported to bind and regulate the transcriptional activity of several nuclear receptors. Of candidates, upon inhibition of the nuclear exportin, SQSTM1 and tumor suppressor TP53 emerge to be recruited to promyelocytic leukemia protein nuclear bodies (PML-NBs), which are involved in DNA repair, TP53-associated cell cycle arrest, and apoptosis. As the guardian of the genome, TP53 plays a central role in genome stability, acting primarily by inducing the expression of the DNA repair proteins. Therefore, It would be interesting to determine whether SQSTM1 regulates DNA repair response by forming a transcriptional complex with Tp53. Identifying such transcription factors controlled by SQSTM1 will have far-reaching significance in tumor immunobiology, by discovering new therapeutic target that may rescue PD-L1 expression for cold refractory cancer.

Collectively, the inventors provide the first evidence that SQSTM1 contributes to the tumor cross-sensitivity to DDA, ICI and ICI/DDA via the reactivation of IFN pathway. This proposes SQSTM1 as a predictive biomarker for both ICI, DDA as monotherapies and combo. Remarkedly, regardless of therapy used (radiotherapy, inducers of immunogenic cell death inducers, and non-ICD chemotherapies), the inventors' data also reveal a novel and global role of SQSTM1 in the induction of HLA-B/PD-L1 expression by DNA damaging agents.

### Translation of the Inventors' Results to Individualized Treatment Decisions

Treatment decisions in immuno-oncology are increasingly challenging with the introduction of ICI combination. No single marker has yet emerged that accurately predicts a therapy response nor tailors optimal combinations. A promising biomarker is suggested here by SQSTM1, which predicts ICI/DDA response and could guide treatment decisions. The inventors' objectives are now to validate SQSTM1 as a first predictive biomarker of ICIs/DDA response that can be rapidly translated to the clinic and ultimately be used as a companion test of the FDA-approved combination strategy. Indeed, the inventors' findings have four potentially high-impact clinical implications:

(1) They recommend concurrent combination therapy rather than sequential monotherapies as the inventors have identified cross-resistance mechanisms that downregulate SQSTM1 for tumor evasion to ICIs and DDA.

(2) They could guide treatment decisions between two different ICI combinations. By linking SQSTM1 to antigen presentation, and PD-L1 expression, they could re-define the patient selection strategy by further restricting ICIs, DDA or ICIs/Cisplatin therapies to patients overexpressing SQSTM1, thereby increasing response rates;

(3) They could also provide the rationale to treat a substantial number of previously excluded patients harboring KRAS and STK11 mutations but overexpressing SQSTM1 as the inventors have provided using A549 cells the proof of principle that Cisplatin, and Docetaxel, can prime this cold subgroup to immunotherapy.

(4) They offer the new therapeutic opportunity of treating those patients with a low-level SQSTM1-expressing LUAD with ICI/MTA (microtubule targeting agents: Docetaxel, Paclitaxel) combo.

Designing a trial around SQSTM1 predicting value should be urgently tested with a larger, well-designed cohort to identify groups of patients who should be switched to either an ICI/Cisplatin or an alternate ICI/Docetaxel strategy, ultimately improving ICI efficacy and patient outcomes. If the test for SQSTM1 IHC score is clinically validated, the inventors hope to expand the utilization of ICIs and DDA to the other cold tumors, improving patient outcomes.

### Materials and Methods

### Cell Culture

The majority of experiments presented in this study was performed with the human KRAS G12S/SKT11 Q37* co-mutated NSCLC A549 cells (American Type Tissue Collection, ATCC). All cells were maintained in Dulbecco's-modified minimum essential medium and Ham's F-12 medium (DMEM/F12 Glutamax; Life Technologies) supplemented with 10% fetal bovine serum (Dutcher), 2% Sodium Pyruvate, and 1% Penicillin-Streptomycin (Life technologies). To establish SQSTM1 knockdown stable cell lines, cells were transduced with small hairpin RNAs (shRNAs) lentiviruses that target the mRNA coding sequence of SQSTM1. Two distinct SQSTM1 shRNAs (Sigma, human, NM_003900, SQSTM1 #1, TRCN0000007237, and SQSTM1 #2, TRCN0000007236) were used to minimize sequence-dependent off-target effects. As controls, autophagy was inhibited at the initiation step by ATG5 (Sigma, human, NM_004849, ATG5 #1, TRCN0000151963) or ATG7 shRNA (Sigma, human, NM_006395, TRCN0000007584). For this purpose, the targeted and control (Sigma; SHC002V) shRNA lentivirus were transduced into the cells. ShRNA-mediated protein downregulation was controlled by qRT-PCR or immunoblotting with specific primers and antibodies (see for shRNA, primer, and antibody details the supplemental Tables 1 to 3).

TABLE 1

| Target | NCBI | | | | | TRC | |
|---|---|---|---|---|---|---|---|
| Gene | Ref | Clone ID | Pathways | Supplier | | Number | Target sequence |
| SQS | NM_ | NM_ | Signaling | SIGMA | SQSTM1 | TRCN0000 | GATCAACTTCAATGC |
| TM1 | 003009 | 003900.2- | scaffold | | #1 | 007237 | CCAGAGGTTTTT |
| | | 788s1c | | | | | (SEQ ID NO: 3) |
| | | 1 | | | | | |
| | | NM_ | Autophagy | SIGMA | SQSTM1 | TRCN0000 | GTTCTCTTTAATGTA |
| | | 003900.2- | receptor | | #2 | 007236 | GATTCGGTTTTT |
| | | 325s1c1 | | | | | (SEQ ID NO: 4) |
| ATG | NM_ | NM_ | Autophagy | SIGMA | ATG5 | TRCN0000 | CCGGCCTGAACAGA |
| 5 | 004849 | 004849.1- | | | | 151963 | ATCATCCTTAACTCG |
| | | 1170s1 | | | | | AGTTAAGGATGATTC |
| | | c1 | | | | | TGTTCAGGTTTTTTG |
| | | | | | | | (SEQ ID NO: 5) |
| ATG | NM_ | NM_ | | SIGMA | ATG7 | TRCN0000 | CCGGGCCTGC |
| 7 | 006395 | 006395.1- | | | | 007584 | TGAGGAGCTC |
| | | 2173s1 | | | | | TCCATCTCG |
| | | c1 | | | | | (SEQ ID NO: 6) |
| Control | | | Non-target | SIGMA | SHC002 | | AGTTGGTGCT |
| ol | | | shRNA | | V | | CTTCATCTTG |
| | | | | | | | TTGTTTTT |
| | | | | | | | (SEQ ID NO: 7) |

TABLE 2

| | Gene | Strand | Sequence 5′−>3′ | SEQ ID NO: |
|---|---|---|---|---|
| Cell cycle arrest | p21 | F | TGAGCCGCGA CTGTGATG | SEQ ID |
| | | | | NO: 8 |
| | | R | GTC TCG GTG ACA AAG TCG | SEQ ID |
| | | | AAG TT | NO: 9 |
| Epigenetic | DNMT1 | F | GCTGAGGCATGAGAATCGCTT | SEQ ID |
| regulators | | | GAA | NO: 10 |
| | | R | GGAGGATCGCTTGAGGTTAGG | SEQ ID |
| | | | AGTT | NO: 11 |
| | DNMT3a | F | GGGACAAGAATGCCACCAAA | SEQ ID |
| | | | | NO: 12 |
| | | R | GAACCACATGACCCAGCG | SEQ ID |
| | | | | NO: 13 |
| | DNMT3b | F | AATGTGAATCCAGCCAGGAAA | SEQ ID |
| | | | GGC | NO: 14 |
| | | R | ACTGGATTACACTCCAGGAAC | SEQ ID |
| | | | CGT | NO: 15 |
| | SETDB1 | F | CTGTCATGTCAGGTCCAAAGC | SEQ ID |
| | | | C | NO: 16 |
| | a | R | TTCCCTCACATTGTCATTGAGC | SEQ ID |
| | | | C | NO: 17 |
| | HEZH2 | F | GACCTCTGTCTTACTTGTGGA | SEQ ID |
| | | | GC | NO: 18 |
| | | R | CGTCAGATGGTGCCAGCAATA | SEQ ID |
| | | | G | NO: 19 |
| Interleukins | IL28 | F | CTCAGGTTGCATGACTGGTGG | SEQ ID |
| | | | | NO: 20 |
| | | R | GAGGCCTCTGTCACCTTCAAC | SEQ ID |
| | | | | NO: 21 |
| | IL29 | F | GGACGCCTTGGAAGAGTCACT | SEQ ID |
| | | | | NO: 22 |
| | | R | CAGGTCCCAATTC | SEQ ID |
| | | | | NO: 23 |
| | IFNa | F | GACTCCATCTTGGCTGTGA | SEQ ID |
| | | | | NO: 24 |
| | | R | TGATTTCTGCTCTGACAACCT | SEQ ID |
| | | | | NO: 25 |
| | IFNb | F | TGCATCTTCTCCGTCATCTC | SEQ ID |
| | | | | NO: 26 |
| | | R | TAGCAGCCGACACCAGCCTG | SEQ ID |
| | | | | NO: 27 |
| | CXCL10 | F | TGAATCCAGAATCGAAGGCCA | SEQ ID |
| | | | | NO: 28 |
| | | R | TGCATCGATTTTGCTCCCCT | SEQ ID |
| | | | | NO: 29 |
| ISG | PD-L1 | F | GGGCATTCCAGAAAGATGAG | SEQ ID |
| | | | | NO: 30 |
| | | R | CCGTGACAGTAAATGCGTTC | SEQ ID |
| | | | | NO: 31 |
| | HLA-B | F | CCCTCACCCTGAGATGGGAG | SEQ ID |
| | | | | NO: 32 |
| | | R | AGCTCCGATGACCACAACTG | SEQ ID |
| | | | | NO: 33 |
| | OAS1 | F | AGGAAAGGTGCTTCCGAGGTA | SEQ ID |
| | | | G | NO: 34 |
| | | R | GGACTGAGGAAGACAACCAGG | SEQ ID |
| | | | T | NO: 35 |
| Housekeeping | RPLP0 | F | GCATCAGTACCCCATTCTATCA | SEQ ID |
| Controls | | | T | NO: 36 |
| | | R | AGGTGTAATCCGTCTCCACAG | SEQ ID |
| | | | A | NO: 37 |
| | GUSB | F | CAGTTCCCTCCAGCTTCAATG | SEQ ID |
| | | | | NO: 38 |
| | | R | ACCCAGCCGACAAAATGC | SEQ ID |
| | | | | NO: 39 |

TABLE 3

| | | | | | | MW | WCL | |
|---|---|---|---|---|---|---|---|---|
| | Antibodies | Genecard | Supplier | Ref | Source | (kDa) | (μg) | Dilution |
| DNA damage | CHK2 | Checkpoint Kinase | CST | 6334T | Rabbit | 62 | 50 | 1000× |
| response | P-CHK2 | Phospho-Checkpoint | CST | 2661T | Rabbit | 62 | 50 | 1000× |
| | (Thr68) | Kinase | | | | | | |
| Cell cycle | P21 | Cyclin Dependent | CST | 2947S | Rabbit | 27 | 25 | 1000× |
| arrest | | Kinase Inhibitor 1A | | | | | | |
| | TBK1 | TANK Binding | CST | 3504T | Rabbit | 84 | 25 | 1000× |
| | | Kinase 1 | | | | | | |
| | p-TBK1 | Phospho-TANK | CST | 5483T | Rabbit | 84 | 25 | 1000× |
| | (Ser172) | Binding Kinase 1 | | | | | | |
| | STAT1 | Signal Transducer | CST | 9172P | Rabbit | 91 | 25 | 1000× |
| | | And Activator Of | | | | | | |
| | | Transcription 1 | | | | | | |
| | P-STAT1 | Phospho-Signal | CST | 9167S | Rabbit | 91 | 25 | 1000× |
| | (Tyr701) | Transducer And | | | | | | |
| | | Activator Of | | | | | | |
| | | Transcription 1 | | | | | | |
| | SQSTM1 | Sequestosome 1 | BD | 610833 | Mouse | 62 | 5 | 2000× |
| | | | Transduction | | | | | |
| | PD-L1 | CD274 Molecule | CST | 86744S | Rabbit | 40-50 | 10 | 1000× |
| | HLA-B | Major | Abcam | 193415 | Rabbit | 43 | 10 | 2000× |
| | | Histocompatibility | | | | | | |
| | | Complex, Class I, B | | | | | | |
| Loading | Tubulin-α | Tubulin Alpha 1a | Sigma | T9026 | Mouse | 50-55 | 5 | 10.000× |
| controls for | Actin clone | Actin | Sigma | A3853 | Mouse | 42 | 5 | 10.000× |
| WB | AC-40 | | | | | | | |

| | Antibodies immunohistochim | | | | | | |
|---|---|---|---|---|---|---|---|
| | Antibodies | Genecard | Clone | Supplier | Ref | Source | Dilution |
| DNA damage | 53BP1 | Tumor Protein P53 | | CST | 4937 | Rabbit | 200× |
| response | | Binding Protein 1 | | | | | |
| | SQSTM1 | Sequestosome 1 | | BD | 610833 | Mouse | 400× |
| | | | | Transduction | | | |
| | PD-L1 | CD274 Molecule | 22C3 | Dako | M365329 | | 50× |
| | cytotoxic | CD8 | SP57 | Ventana | | Mouse | prediluted |
| | T cell | | | | | | |

Antibodies FACS

| Antibodies | Clone | Format | Isotype | Supplier | Ref | Ref Isotype |
|---|---|---|---|---|---|---|
| PD-L1 | 10F.9G2 | BV650 | Mouse | Biolegend | 329739 | 400351 |
| | | | IgG2b, κ | | | |
| HLA-A, B, C | W6/32 | PE | Mouse | Biolegend | 311405 | 400211 |
| | | | IgG2a, κ | | | |
| Live/Dead | | PercPCy5.5 | | Invitrogen | | |

### Cell Treatments

For all experiments, cells were seeded in 6-well plates or 60 mm culture dishes and incubated until they reached 60-70% confluency. Cells were then treated with Cisplatin (Cis, 10 μM) in fresh DMEM supplemented with 1% FBS for the indicated times. Under similar conditions, the inventors extended the inventors' study to other chemotherapeutic agents known to induce DNA damage and immunogenic cell death (ICD) such as anthracyclines Doxorubicin (Dox, 0.5 μM), Oxaliplatin (Ox, 1.4 μM), and taxanes (Docetaxel, D, 5 nM and Paclitaxel, P, 3 nM, Table 4). Drug concentrations were chosen through dose-response curves based on the peak of blood concentration reached by each drug [IC50s: Shc A549 (Cis=24 μM+/−3.7, Dox=364 nM+/−3, Ox=2.61 μM+/−0.9, D=364 nM+/−3, P=1.4 nM+/−0.27), ShSQSTM1 A549 (Cis=47.7 μM+/−5, Dox=486 nM+/−85, Ox=4.51 μM+/−1, D=21.3 nM+/−10, P=3.7M+/−0.7)].

As controls, to ensure that the DDA resistance of SQSTM1-depleted cells was not related to drug efflux, cells were irradiated at 10 Gy using the Faxitron X-ray system (model 43855 F with CP 160 Option; Faxitron Bioptics), operating at 160 kV/6.3 mA. Immediately after irradiation, cells were trypsinized and reseeded in 6-well plates with fresh 10% FBS medium. All chemotherapeutics were obtained from Antoine-Lacassagne Cancer Centre.

Where indicated, MRT67307 (TBK1 inhibitor, 10 μM, Tocris) or Ruxolitinib (JAK1/JAK2 inhibitor, 5 μM, Tocris) was added to the 1% FBS medium for 90 minutes before the addition of Cisplatin (10 μM). As a positive control, cells were treated with IFNγ (50 ng/ml, 24 h, C-60724 Promokine).

All pharmacological inhibitors at the doses studied here failed to induce cell death for 18-24 h. The ability of DNA damage agents to activate the innate defense was checked by the downstream induction of IFNs, IFN-mediated signaling, expression of HLA-B, and PD-L1.

Relative Quantification of mRNA Levels

Total RNA was purified from cells using the TRI Reagent (TR 118, MRC) and RNeasy Mini Kit (#74106, Qiagen). 600 ng of total RNA was treated with DNAse I (18068015, Invivogen) and reverse transcribed using the SuperScript III Reverse Transcriptase (Life Technologies). Quantitative real-time polymerase chain reaction analysis was performed on equal amounts of cDNA using the SYBR Green master mix and an Applied StepOne Plus PCR system (Life technologies). The primers of IFN response genes used are listed in the Table 2. Relative gene expression changes were quantified using the 2 [−ΔΔCt] method and normalized to the housekeeping gene RPLP0 compared to the untreated cell samples.

### Western Blotting

After treatments, the cells are washed in PBS, and the whole-cell lysates (WCL) were extracted with TR3 lysis buffer containing 3% SDS; 10% glycerol, 10 mM Na2PO4, with protease and phosphatase inhibitors cocktail (1 (mM Na3VO4, 10 mM β-glycerophosphate, 10 mM NaF and 1:25, Complete™) and sonicated. WCL (5-40 μg) were analyzed by western blotting with antibodies that specifically recognize SQSTM1, PD-L1, HLA-B, DNA damage, cell cycle arrest, and TBK1 and JAK signaling pathways (Table 3), as previously described. Tubulin, Actin (#A3853, Sigma), and HSP90 (clone C45G5, #4877S, Cell Signaling Technology) were used as loading controls. After washing, the presence of primary antibodies was revealed with horseradish peroxidase-conjugated-anti-mouse (1:6,000; sc-2005; Santa Cruz) or-anti-rabbit (1:10,000; sc-45040; Santa Cruz) and visualized with the Enhanced Chemiluminescence detection system (Perkin Elmer).

### Flow Cytometry Analysis

Cell-surface expression of PD-L1 was examined using flow cytometry. After treatment 10 μM Cisplatin for the indicated times, cells were harvested in 2.5 mM EDTA-PBS without trypsinization, labeled with anti-PD-L1 antibodies (CD274, brilliant violet 650 conjugate, #329740, Biolegend), or anti-isotype antibodies (brilliant violet 650 conjugate, #400351, Biolegend). Flow cytometry analysis was performed on a Cytoflex flow cytometer (10,000 cells, Cytoflex software). The MFI (PD-L1-isotype) is calculated as the MFI (PD-L1) is subtracted by the MFI (isotype control).

### Dataset Analysis

The cBio cancer genomics portal (http://www.cbioportal.org/public-portal/) and the Phantasus software (https://artyomovlab.wustl.edu/phantasus/) were used to mine data from The Cancer Genome Atlas (TCGA) PanCancer Atlas and the Cancer Cell-line Encyclopedia (CCLE). Several datasets (RNAseq, DNA microarrays) of cancer patients treated with chemotherapy, radiotherapy, and immunotherapy were downloaded from the Gene Expression Omnibus (GEO Database, https://www.ncbi.nlm.nih.gov/gds). The signatures of

T-lymphocyte infiltration, DNA damage response, IFN (C2CGP, C2 reactome, C5BP, and “hallmarks”) in each tumor were correlated to gene expression of CD274/PD-L1, CD8A/B, HLA- and SQSTM1 expression by Gene Set Enrichment Analysis (GSEA) and ssGSEA analyses. For a complete list of the TCGA cancer-type abbreviations, please see https://gdc.cancer.gov/resources-tcga-users/tcga-code-tables/tcga-study-abbreviations.

### Patient Cohort

The cohort of patients with lung adenocarcinoma (LUAD) was conducted at the Laboratory of Clinical and Experimental Pathology (Nice, France), University Côthe d′Azur, between the first January of 2010 and the first April of 2018 was investigated. The study was performed according to the REMARK-guidelines and was approved by the Ethics Commission of the Nice University Hospital, which waived the requirement for written informed consent. Initially, 468 patients met the inclusion criteria of the LUAD diagnosis according to the pathology records. Immunohistochemical stainings for p40 and TTF-1 definitively confirmed the glandular differentiation of the tumors included in the study. Furthermore, the slides of all tumors were reevaluated regarding stage-relevant characteristics (such as pleural invasion). All the tumors were re-staged according to the 8th edition of the UICC TNM-classification. The case collection finally comprised 287 tumors from early stages (I-IIIA) and 181 tumors from late stages (IIIB-IV). Adjuvant chemotherapy and/or radiotherapy was administered in 70/181 (39%) patients, EGFR TKIs was administered in 18/181 (10%), and first (pembrolizumab) or second-line (nivolumab) immunotherapy was administered in 37/181 (20%) and 41/181 (23%) patients, respectively. 15/181 (8%) patients died before any adjuvant treatment.

### Immunohistochemical Staining and Scoring

Immunohistochemical staining for p62/SQSTM1, PD-L1/CD274, and CD8 were performed on 4 μm sections using an automated Ultra Ventana (Ventana, Tucson, AZ), as described before for SQSTM1 (dilution 1/400, BD Transduction Laboratories™), PD-L1 (clone 22C3, dilution 1/50, Dako, Inc.) and CD8 (cytotoxic T cell; clone SP57, prediluted; Ventana) (Table 3) and were used according to the instructions of the manufacturer. Scoring of immunohistochemical staining patterns for SQSTM1 detected in various subcellular components of the tumor cells was performed across all whole sections: dot-like staining was scored from 0 to 3 as follows: score 0-no dots or barely dots visible in <5% of the tumor cells, score 1-dots in 5-25% of the tumor cells, score 2-dots in 25-75% of the tumor cells, score 3-dots in >75% of the tumor cells. SQSTM1 cytoplasmic staining was scored from 0 to 3 as follows: score 0-no or faint staining, score 1-weak staining, score 2-moderate staining visible, and score 3-strong staining. SQSTM1 nuclear immunohistochemical staining was scored from 0 to 1 as follows: score 0-nuclear staining visible in <10% of nuclei and score 1-nuclear staining visible in >10% of nuclei. Scoring was performed by two experienced pathologists (VH and PH) at 40× objective magnification. For correlation with clinicopathological features, the immunohistochemical scores were then further categorized as either low or high, and according to the single values' prognostic value. Dot-like and cytoplasmic SQSTM1 staining was categorized as low for scores 0-1 and high for scores 2-3. Combined dot-like and cytoplasmic staining for SQSTM1 was classified as low for a sum score of 0-2 and high for a sum score of 3 and greater of the raw values, showing the best prognostic discrimination. SQSTM1 nuclear staining score 0 was classified as low, and score 1 as high. PD-L1 positive tumor cells were counted, and one cut-off was used (>50% PD-L1 positive tumor cells). Intra-tumoral CD8 positive cells were counted and tumors were classified as no (−), low (+), moderate (++) and high (+++) tumor expressers.

Subclassification according to SQSTM1, PD-L1, and CD8 status. A combination of SQSTM1 dot-like/cytoplasmic, PD-L1 and CD8 staining stratified the cases into 3 subtypes: low SQSTM1 dot-like-cytoplasmic/low PD-L1/low CD8 stainings (LLL); high SQSTM1 dot-like-cytoplasmic/high PD-L1/high CD8 (HHH); and high SQSTM1 dot-like-cytoplasmic/low PD-L1; high CD8 stainings (HLH).

### Statistical Analysis

For statistical analysis, GraphPad Prism 6 software was used to analyze data. Group comparisons were performed using crosstabs, unpaired nonparametric T test, χ2-tests, ANOVA, and Fisher's exact tests. Values are presented as means and standard deviations (SD). P<0.05 was set as achieving statistical significance. Survival analysis encompassed time to recurrence (TTR) was measured from the day of resection to locoregional or metastatic recurrence or disease-specific death. Disease-specific survival (DSS) was determined from the time of diagnosis to disease-specific death. Overall survival (OS) and disease-free survival (DFS) were calculated. Kaplan-Meier curves and log-rank tests were used for univariate survival analysis. For multivariate analysis, Cox regression analysis was used. The significance level for all statistical tests was set for a p-value of <0.05.

### Example 2: SQSTM1 is an Immunotherapy Predictive Biomarker in Advanced Melanoma

The inventors validated the interest of SQSTM1 as a predictive biomarker in advanced skin melanoma (SKCM), another immunogenic solid tumor that benefits from anti-PD1immunotherapy.

### 1. Rationale

Metastatic or advanced melanoma is a fatal skin cancer, with a 5-y survival rate of less than 30% until now. The development of the Immune Checkpoint Inhibitors (ICIs) targeting Programmed Death-1 (PD-1) and its ligand PD-L1 represents a true paradigm shift with a 52% increase in the 5-y median overall survival. However, durable response to ICIs is limited to only a subset of patients, whereas 40% of the patients do not respond to ICIs in monotherapy.

In most clinical trials, the expression of PD-L1 assessed by immunohistochemistry does not allow the selection of responding patients (Robert C et al., 2015). We provide the first evidence that non-small cell lung carcinoma cancer patients with increased levels of SQSTM1 have a better response than those with low levels, highly indicating SQSTM1 as a predictor of response to ICIs. Here, the objective of our study was to correlate the expression of SQSTM1 in melanoma cells combined with the quantification of intratumoral PD-L1 and of the infiltration of CD8+ T lymphocytes to ICI response, to determining whether the SQSTM1 evaluation could be an effective predictive biomarker in patients with relapsed or metastatic melanoma.

### 2. Materials and Methods

a) Patients and tissue samples (Ilié M, et al. Oncoimmunology. 2021 Mar. 19; 10 (1): 1901446). This retrospective cohort included 125 patients with consecutive primary cutaneous malignant melanoma diagnosed between July 2013 and February 2017 and treated at the Department of Dermatology, University of Nice, Archet 2 Hospital (Nice, France). The patients initially diagnosed with stage I-II melanoma were enrolled in the study at the time of the regional or distant metastatic relapse. The availability of histological material from the metastasis and the presence of an informed signed consent were required criteria to include a case in the study.

Out of the 125 patients, 91 (73%) presented with regional metastases (35 in transit and 56 lymph node metastases) and 34 (27%) with distant metastases (19 lung and 15 subcutaneous metastasis sites).

Two groups of patients were distinguished in this study: a group of 58 patients (46%) who received at least one treatment of immunotherapy (anti-PD-1 inhibitors-pembrolizumab/nivolumab and/or anti-CTLA4) and a group of 67 patients (53%) who did not receive immunotherapy treatment, albeit some had other treatments (chemotherapy or targeted therapies with anti-BRAF and anti-MEK agents)

All tumor specimens were used with the informed signed consent from the patients. The study was approved by the local ethics committee (Human Research Ethics Committee, Nice University Hospital Center/hospital-related Biobank BB-0033-00025; http://www.biobank-cotedazur.fr/) and was performed following the guidelines of the Declaration of Helsinki.

b) Immunohistochemistry (IHC) was performed according to standard protocols using SQSTM1 antibodies, as previously described. Briefly, Formalin-fixed paraffin-embedded (FFPE) serial 4 μm tissue sections were freshly cut, deparaffinized, pre-treated, and stained with monoclonal antibodies directed against SQSTM1 (BD Transduction Laboratories™, 610833) on a BenchMark ULTRA autostainer (Ventana Medical Systems, Tucson, AZ, USA). Stains were detected using anti-immunoglobulin-coupled horseradish peroxidase with 3,3-diaminobenzidine (DAB, OptiView Kit, Roche Diagnostics, Ventana, catalog no. 760-700) as substrate. Nuclear counterstaining was performed with Mayer hematoxylin. Each IHC run contained a positive control, and a negative Ab control (buffer, no primary Ab).

c) Evaluation of immunohistochemistry. Inter- and intra-observer variability. The immunohistochemical scoring for SQSTM1 (cytoplasmic and/or nuclear) was examined for intra- and interobserver variabilities. Two pathologists independently evaluated immunohistochemical staining results without knowledge of clinicopathologic data. The inter-observer agreement of the two pathologists was high (α=0.97). The intra-observer agreement of the scoring also showed a high concordance. Discrepancy results were resolved using a multiheaded microscope.

d) IHC scoring. The intensity, percentage, and subcellular localization of the immunohistochemical staining of each case were recorded. Staining omitting the primary antibody was performed as a negative control. The intensity and percentage of positively stained cells were scanned at a low-powered field (×100) and then evaluated at a high-powered field (×400). SQSTM1 staining was identified in cytoplasms and nuclei. The intensity of SQSTM1 staining was recorded as 0, 1, 2, and 3, referring to negative, weak, moderate, and strong staining, respectively (see below). The percentage of SQSTM1 positive cells was recorded from 0 to 100%. The results of staining were scored using a quick (Q) score, which was obtained by multiplying the percentage of positive cells (P) by the intensity (I) (Q=P×I; maximum=300; Charafe-Jauffret et al., 2004). The median values of the Q scores of melanoma were used as cutoff points to classify cancer as exhibiting ‘low expression’ and ‘high expression. The χ2 test or Student's t-test was used to investigate the difference between groups. FIG. 31.

### 3. Results

SQSTM1 is a nucleocytoplasmic shuttling protein but the association of its subcellular localization with skin carcinogenesis and the response to immunotherapy was not documented so far.

The inventors retrospectively evaluated the association of SQSTM1 expressed in melanoma cells in combination with intratumoral and CD8+ T lymphocytes, as detected by immunohistochemistry and quantified by digital analysis clinicopathological features and overall survival (OS) among 58 patients treated with ICIs.

They found that melanoma displayed stronger SQSTM1 expression than that normal skin. Of ICI-treated melanoma, the non-responder melanoma exhibited limited cytoplasmic SQSTM1 staining. In contrast, the responder melanoma exhibited the highest increase in cytoplasmic and nuclear SQSTM1 expression. The differences in cytoplasmic and nuclear expression between the two groups (non-responders and responders) were statistically significant (p-value=0.00026). Altogether, these findings provided the first evidence of the interest of the nuclear and cytoplasmic SQSTM1 staining as an ICI predictive biomarker of skin melanoma.

### Example 3: SQSTM1 is a Circulating Biomarker in Liquid Biopsy for Immunotherapy Stratification in Lung Adenocarcinoma (LUAD)

The inventors also evaluated the predictive value of SQSTM1 as a non-invasive circulating biomarker in liquid biopsy for improved immunotherapy stratification in LUAD.

### 1. Rationale

Obtaining adequate tumor tissue for molecular testing is more and more challenging in patients with advanced or metastatic disease for whom tissue biopsies are inaccessible, extremely difficult to perform or if a low percentage of tumor cells are present (leading to a low amount of extracted nucleic acids for molecular testing and/or for a robust assessment of PD-L1 expression). Thus, an unmet diagnostic need urges non-invasive approaches that identify patients who may benefit from immunotherapy. In recent years, i) the inventors used a protocol to isolate specifically Circulating Tumor Cells (CTCs) from the blood of lung cancer patients using the ISET® platform (Isolation by SizE of Tumor cells, Hofman V 2011a/b), ii) the inventors successfully reported the overexpression of PD-L1 on circulating tumor cells (CTCs) in the blood of some but not all patients with advanced lung cancer (NSCLC); iii) the inventors correlated the PD-L1 expression in CTCs and matched lung biopsies; iv) however, the inaccuracies of PD-L1 false positive and false negative staining remain (Ilie M, et al. Ann Oncol. 2018 Jan. 1; 29 (1): 193-199).

In NSCLC patients, SQSTM1 expression in tumor biopsies correlated with improved benefit to the PD-L1 inhibitor. The inventors thus investigated the utility of CTCs as non-invasive surrogates for the tumor's PD-L1 status by evaluating the prevalence of PD-L1 and SQSTM1 expressions in blood samples using the ISET platform SQSTM1 expression in both CTCs, and matched tumor tissue in a cohort of 40 advanced-stage NSCLC patients.

### 2. Methods

a) CTC capture. CTC capture was performed by a method that combines size-based filtration with cytopathological evaluation (ISET® technology). Briefly, blood samples were drawn into K3EDTA or blood collection tubes (BCT) (Streck), and filtered by the Isolation by SizE of Tumor (ISET® system, Rarecells, Paris, France) for the capture of CTC, according to the manufacturer's recommendations (llie M, et al. Ann Oncol. 2018 Jan. 1; 29 (1): 193-199). The filters were analyzed for the presence of circulating non-hematological cells with malignant (CNHC-MF) or uncertain (CNHC-UMF) features.

b) SQSTM1 expression on ISET filters. Samples that presented CTC detection (≥2 CNHC-MF and/or CNHC-UMF) were selected for further analysis of SQSTM1 expression by immunocytochemistry on three unstained ISET filter spots, as follows: after 2 min of rehydration with Reaction Buffer 10× (catalog #950-300; Ventana), filters were placed on positively charged glass slides in the BenchMark ULTRA autostainer (Ventana) and followed the SQSTM1 staining protocol as for IHC (as described above).

The SQSTM1 ICC analysis assessed the cytoplasmic and nuclear expression of SQSTM1 and scored the percentage of CTCs and WBCs expressing SQSTM1. Results from blood samples and matched-tumor tissue were blinded until study completion.

### 3. Results

The inventors isolated CTCs from LUAD patient blood samples using ISET® and successfully detected SQSTM1 by immunocytochemical staining. This proof-of-concept study demonstrated the predictive value of the CTC/SQSTM1 assay as a non-invasive real-time liquid biopsy for immunotherapy response patient stratification.

1-13. (canceled)

14. A method for predicting in vitro the resistance of a tumor to a therapy, said therapy being an immunotherapy against checkpoint inhibitors or ICI or a combination of an ICI and a chemotherapy, said method comprising:
evaluating the presence or the absence or the amount of an SQSTM1/p62 protein in a biological sample originating from the tumor,
comparing the presence, absence or the amount of the SQSTM1/p62 protein with the amount of the SQSTM1/p62 protein in a control sample,
concluding that
the tumor will be likely to be resistant to the therapy when SQSTM1/p62 protein is absent or lower than or equal to the amount obtained in the control sample in the said biological sample, and
the tumor will be likely to be sensitive to the therapy when SQSTM1/p62 protein is present or higher than a control level in the said biological sample.

15. The method, according to claim 14, wherein the presence or absence of the SQSTM1/p62 protein is evaluated in situ in the biological sample.

16. A method for predicting the survival rate of a patient afflicted by a tumor, said method comprising:
evaluating the presence or the absence or the amount of an SQSTM1/p62 protein in a biological sample originating from the tumor,
comparing the presence, absence or the amount of the SQSTM1/p62 protein with the amount of the SQSTM1/p62 protein evaluated in a control sample,
concluding that
when SQSTM1/p62 in the said biological sample is absent or lower than or equal to the amount obtained in the control sample, then the patient will have a survival rate higher than 80% after 5 years, and
when SQSTM1/p62 in the said biological sample is present or higher than the amount obtained in the control sample, then the patient will have a survival rate lower than 70% after 5 years.

17. The method according to claim 16, wherein the presence, absence or amount of
a PD-L1 protein, and
CD8+ T lymphocytes
are evaluated concomitantly to the presence or the absence or the amount of an SQSTM1/p62 protein, and compared with the presence, absence or the amount of the respective PD-L1 protein and CD8+ T lymphocytes evaluated in a control sample,
and wherein
when SQSTM1/p62 protein, PD-L1 protein, and CD8+ T lymphocytes, in the said biological sample, are present or higher than the amount obtained in the control sample, then the patient will have a survival rate lower than 50% after 5 years.

18. A method for predicting the survival rate of a patient afflicted by a tumor and treated with an ICI or a combination of an ICI and a chemotherapy, said method comprising:
evaluating the presence or the absence or the amount of an SQSTM1/p62 protein in a biological sample originating from the tumor,
comparing the presence, absence or the amount of the SQSTM1/p62 protein with the amount of the SQSTM1/p62 protein evaluated in a control sample,
concluding that
when SQSTM1/p62 in the said biological sample is absent or lower than or equal to the amount obtained in the control sample, then the patient will have a survival rate lower than or equal than 10% after 20 months of treatment, and
when SQSTM1/p62 in the said biological sample is present or higher than the amount obtained in the control sample, then the patient will have a survival rate equal to or higher than 50% after 20 months of treatment.

19. A Method for treating a pathology involving inflammation, comprising the administration to an individual in need therefore of an effective amount of a composition comprising:
an SQSTM1/p62 protein; or
a nucleic acid molecule coding for said SQSTM1/p62 protein;
along with an immunotherapeutic antibody directed against a checkpoint inhibitor or a combination of a chemotherapeutic agent and an immunotherapeutic antibody directed against a checkpoint inhibitor.

20. The method according to claim 19, wherein said pathology involving inflammation are cancers.

21. A kit comprising:
an SQSTM1 protein,
an antibody useful for inducing immunotherapy directed against a checkpoint inhibitor, and
a chemotherapeutic agent.

22. The kit according to claim 21, wherein said antibody is an anti-PD-L1 antibody, an anti-PD-1 antibody, or an anti-CTLA-4 antibody.

23. A method for treating tumors that do not express SQSTM1/p62 protein, or tumors that express SQSTM1/p62 protein at a level lower than the level of SQSTM1/p62 protein in a control tissue comprising the administration to an individual in need therefore of an effective amount of a composition comprising an immunotherapeutic ICI compound in association with taxanes.

24. A method for treating tumors that express SQSTM1/p62 protein, or tumors that express SQSTM1/p62 protein at a level higher than the level of SQSTM1/p62 protein in a control tissue comprising the administration to an individual in need therefore of an effective amount of a composition comprising an immunotherapeutic ICI compound in association with a DNA damage-inducing agent.

25. The method according to claim 14, wherein said SQSTM1/p62 protein comprises or consists of the amino acid sequence as set forth in SEQ ID NO: 1.

26. The method according to claim 15, wherein the biological sample is a tissue biopsy or a liquid biopsy.

27. The method according to claim 20, wherein the cancer is one of primary tumors or metastatic tumors.

28. The method according to claim 20, wherein the primary tumors or metastatic tumors are chosen from lung cancers, kidneys cancers, bladder cancers, head neck cancers uterine cancer, melanoma, Hodgkin's lymphoma, Large B cell lymphoma Merkel disease, hepatocellular carcinoma, and gastro-intestinal cancers, and gastro-intestinal cancer with minisatellite instability.

29. The kit according to claim 21, wherein said chemotherapeutic agent is either a chemotherapeutic containing platin compounds, or a Paclitaxel or Docetaxel compound, or a radiotherapy.


SEQUENCE LISTING (39 sequences)

SEQ ID NO: 1 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 2 - DNA, 2840 bp, Homo sapiens
acggcccgttttccgccagctcgccgctcgctatggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgaggggcgccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtgaccacttttgcccacctcttctgcgtgcccctcttctgtctcatagttgtgttaagcttgcgtagaattgcaggtctctgtacgggccagtttctctgccttcttccaggatcaggggttagggtgcaagaagccatttagggcagcaaaacaagtgacatgaagggagggtccctgtgtgtgtgtgtgctgatgtttcctgggtgccctggctccttgcagcagggctgggcctgcgagacccaaggctcactgcagcgcgctcctgacccctccctgcaggggctacgttagcagcccagcacatagcttgcctaatggctttcactttctcttttgttttaaatgactcataggtccctgacatttagttgattattttctgctacagacctggtacactctgattttagataaagtaagcctaggtgttgtcagcaggcaggctggggaggccagtgttgtgggcttcctgctgggactgagaaggctcacgaagggcatccgcaatgttggtttcactgagagctgcctcctggtctcttcaccactgtagttctctcatttccaaaccatcagctgcttttaaaataagatctctttgtagccatcctgttaaatttgtaaacaatctaattaaatggcatcagcactttaaccaatgacgtttgcatagagagaaatgattgacagtaagtttattgttaatggttcttacagagtatctttaaaagtgccttaggggaaccctgtccctcctaacaagtgtatctcgattaataacctgccagtcccagatcacacatcatcatcgaagtcttccccagttataaagaggtcacatagtcgtgtgggtcgaggattctgtgcctccaggaccaggggcccaccctctgcccagggagtccttgcgtcccatgaggtcttcccgcaaggcctctcagacccagatgtgacggggtgtgtggcccgaggaagctggacagcggcagtgggcctgctgaggccttctcttgaggcctgtgctctgggggtcccttgcttagcctgtgctggaccagctggcctggggtccctctgaagagaccttggctgctcactgtccacatgtgaactttttctaggtggcaggacaaattgcgcccatttagaggatgtggctgtaacctgctggatgggactccatagctccttcccaggacccctcagctccccggcactgcagtctgcagagttctcctggaggcaggggctgctgccttgtttcaccttccatgtcaggccagcctgtccctgaaagagaagatggccatgccctccatgtgtaagaacaatgccagggcccaggaggaccgcctgccctgcctgggccttggctgggcctctggttctgacactttctgctggaagctgtcaggctgggacaggctttgattttgagggttagcaagacaaagcaaataaatgccttccacctcaccgcaaa

SEQ ID NO: 3 - DNA, 27 bp, Artificial sequence
  SQSTM1 fragment
gatcaacttcaatgcccagaggttttt

SEQ ID NO: 4 - DNA, 27 bp, Artificial sequence
  SQSTM1 fragment
gttctctttaatgtagattcggttttt

SEQ ID NO: 5 - DNA, 59 bp, Artificial sequence
  ATG5 fragment
ccggcctgaacagaatcatccttaactcgagttaaggatgattctgttcaggttttttg

SEQ ID NO: 6 - DNA, 29 bp, Artificial sequence
  ATG7 fragment
ccgggcctgctgaggagctctccatctcg

SEQ ID NO: 7 - DNA, 28 bp, Artificial sequence
  controle shRNA
agttggtgctcttcatcttgttgttttt

SEQ ID NO: 8 - DNA, 18 bp, Artificial sequence
  oligonucleotide for PCR reaction
tgagccgcgactgtgatg

SEQ ID NO: 9 - DNA, 23 bp, Artificial sequence
  oligonucleotide for PCR reaction
gtctcggtgacaaagtcgaagtt

SEQ ID NO: 10 - DNA, 24 bp, Artificial sequence
  oligonucleotide for PCR reaction
gctgaggcatgagaatcgcttgaa

SEQ ID NO: 11 - DNA, 25 bp, Artificial sequence
  oligonucleotide for PCR reaction
ggaggatcgcttgaggttaggagtt

SEQ ID NO: 12 - DNA, 20 bp, Artificial sequence
  oligonucleotide for PCR reaction
gggacaagaatgccaccaaa

SEQ ID NO: 13 - DNA, 18 bp, Artificial sequence
  oligonucleotide for PCR reaction
gaaccacatgacccagcg

SEQ ID NO: 14 - DNA, 24 bp, Artificial sequence
  oligonucleotide for PCR reaction
aatgtgaatccagccaggaaaggc

SEQ ID NO: 15 - DNA, 24 bp, Artificial sequence
  oligonucleotide for PCR reaction
actggattacactccaggaaccgt

SEQ ID NO: 16 - DNA, 22 bp, Artificial sequence
  oligonucleotide for PCR reaction
ctgtcatgtcaggtccaaagcc

SEQ ID NO: 17 - DNA, 23 bp, Artificial sequence
  oligonucleotide for PCR reaction
ttccctcacattgtcattgagcc

SEQ ID NO: 18 - DNA, 23 bp, Artificial sequence
  oligonucleotide for PCR reaction
gacctctgtcttacttgtggagc

SEQ ID NO: 19 - DNA, 22 bp, Artificial sequence
  oligonucleotide for PCR reaction
cgtcagatggtgccagcaatag

SEQ ID NO: 20 - DNA, 21 bp, Artificial sequence
  oligonucleotide for PCR reaction
ctcaggttgcatgactggtgg

SEQ ID NO: 21 - DNA, 21 bp, Artificial sequence
  oligonucleotide for PCR reaction
gaggcctctgtcaccttcaac

SEQ ID NO: 22 - DNA, 21 bp, Artificial sequence
  oligonucleotide for PCR reaction
ggacgccttggaagagtcact

SEQ ID NO: 23 - DNA, 13 bp, Artificial sequence
  oligonucleotide for PCR reaction
caggtcccaattc

SEQ ID NO: 24 - DNA, 19 bp, Artificial sequence
  oligonucleotide for PCR reaction
gactccatcttggctgtga

SEQ ID NO: 25 - DNA, 21 bp, Artificial sequence
  oligonucleotide for PCR reaction
tgatttctgctctgacaacct

SEQ ID NO: 26 - DNA, 20 bp, Artificial sequence
  oligonucleotide for PCR reaction
tgcatcttctccgtcatctc

SEQ ID NO: 27 - DNA, 20 bp, Artificial sequence
  oligonucleotide for PCR reaction
tagcagccgacaccagcctg

SEQ ID NO: 28 - DNA, 21 bp, Artificial sequence
  oligonucleotide for PCR reaction
tgaatccagaatcgaaggcca

SEQ ID NO: 29 - DNA, 20 bp, Artificial sequence
  oligonucleotide for PCR reaction
tgcatcgattttgctcccct

SEQ ID NO: 30 - DNA, 20 bp, Artificial sequence
  oligonucleotide for PCR reaction
gggcattccagaaagatgag

SEQ ID NO: 31 - DNA, 20 bp, Artificial sequence
  oligonucleotide for PCR reaction
ccgtgacagtaaatgcgttc

SEQ ID NO: 32 - DNA, 20 bp, Artificial sequence
  oligonucleotide for PCR reaction
ccctcaccctgagatgggag

SEQ ID NO: 33 - DNA, 20 bp, Artificial sequence
  oligonucleotide for PCR reaction
agctccgatgaccacaactg

SEQ ID NO: 34 - DNA, 22 bp, Artificial sequence
  oligonucleotide for PCR reaction
aggaaaggtgcttccgaggtag

SEQ ID NO: 35 - DNA, 22 bp, Artificial sequence
  oligonucleotide for PCR reaction
ggactgaggaagacaaccaggt

SEQ ID NO: 36 - DNA, 23 bp, Artificial sequence
  oligonucleotide for PCR reaction
gcatcagtaccccattctatcat

SEQ ID NO: 37 - DNA, 22 bp, Artificial sequence
  oligonucleotide for PCR reaction
aggtgtaatccgtctccacaga

SEQ ID NO: 38 - DNA, 21 bp, Artificial sequence
  oligonucleotide for PCR reaction
cagttccctccagcttcaatg

SEQ ID NO: 39 - DNA, 18 bp, Artificial sequence
  oligonucleotide for PCR reaction
acccagccgacaaaatgc